(19) United States
(12) Patent Application Publication
(10) Pub. No.: US 2025/0170147 A1
(43) Pub. Date: May 29, 2025

(54) NEUROACTIVE STEROIDS FOR TREATMENT OF GASTROINTESTINAL DISEASES OR CONDITIONS

(71) Applicant: Sage Therapeutics, Inc., Cambridge, MA (US)

(72) Inventor: Stephen Jay Kanes, Villanova, PA (US)

(21) Appl. No.: 18/841,787

(22) PCT Filed: Feb. 15, 2023
(86) PCT No.: PCT/US2023/062630

§ 371 Date: Aug. 27, 2024

Related U.S. Application Data
(60) Provisional application No. 63/442,056, filed on Jan. 30, 2023.
(60) Provisional application No. 63/315,015, filed on Feb. 28, 2022.

(51) Int. Cl. A61K 31/58; A61K 45/06

(52) U.S. Cl. CPC A61K 31/58; A61K 45/06

(57) ABSTRACT

The present disclosure relates to methods of treating a gastrointestinal (GI) disease or condition in a subject in need thereof.

### CROSS REFERENCE TO RELATED APPLICATIONS

This application claims the benefit of U.S. Provisional Application No. 63/315,015, filed on Feb. 28, 2022, and U.S. Provisional Application No. 63/442,056, filed on Jan. 30, 2023. The entire contents of the aforementioned applications are incorporated herein by reference in their entireties.

### FIELD OF THE INVENTION

The present invention relates to methods of treating gastrointestinal diseases or conditions in a subject in need thereof with Compound (1).

### BACKGROUND

Gastrointestinal (GI) disorders or conditions include irritable bowel syndrome (IBS), characterized by a group of symptoms accompanied together that include abdominal pain and changes in the consistency of bowel movements. IBS symptoms include cramping, abdominal pain, bloating, gas, and diarrhea or constipation, or both. IBS has prevalence of about 11-14% of the US adult population, making it one of the most frequently seen disorders in the United States.

IBS has four main subtypes, which are classified based on whether diarrhea, constipation, or both are common: IBS with diarrhea (IBS-d), IBS with constipation (IBS-c), or IBS mixed (IBS-M; mixed constipation and diarrhea). If an IBS patient rarely or never has abnormal stools or does not fit into one of the three aforementioned IBS subtypes, the patient is subtyped as unclassified IBS (IBS-U).

IBS is often diagnosed with the Rome IV Diagnostic Criteria which includes abdominal pain and discomfort lasting on average at least 1 day/week during the 3 months before the diagnosis and associated with 2 or more of the following features: pain and discomfort related to defecation, altering in frequency of defecation, a change in form (appearance) of stool.

The causes of IBS are not clear. There is also no known cure for IBS; treatment of IBS focuses on symptom relief/improvement. Indeed, there are very few FDA approved therapies for the treatment of IBS.

Thus, there remains an unmet medical need for additional, well-tolerated, and effective therapies to treat GI disorders or conditions such as IBS.

### SUMMARY OF THE INVENTION

In one aspect, the present disclosure provides a method of method of treating a gastrointestinal (GI) disease or condition in a subject in need thereof, comprising administering a therapeutically effective amount of Compound (1), or a pharmaceutically acceptable salt thereof,

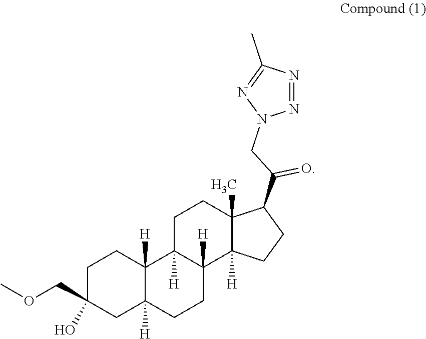

In some embodiments, the GI disease or condition is a non-inflammatory GI disease or condition. In some embodiments, the GI disease or condition is an inflammatory GI disease or condition.

In some embodiments, the GI disease or condition is inflammatory bowel disease (IBD), ulcerative colitis (UC), Crohn's disease (CD), irritable bowel syndrome (IBS), indeterminate colitis (IC), microscopic colitis, collagenous colitis, lymphocytic colitis, incomplete microscopic colitis, segmental colitis associated with diverticula (SCAD), or combinations thereof. In some embodiments, the GI disease or condition is irritable bowel syndrome (IBS). In some embodiments, the IBS is IBS with constipation (IBS-C), IBS with diarrhea (IBS-D), IBS with mixed constipation and diarrhea (IBS-M), IBS with alternating stool pattern (IBS-A), post-infection (PI) IBS, or post diverticulitis IBS.

In some embodiments, the subject has a CNS-related disorder and/or the subject is being treated for a CNS-related disorder. In some embodiments, the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus. In some embodiments, the CNS-related disorder is a mood disorder. In some embodiments, the mood disorder is major depressive disorder or postpartum depression.

In some embodiments, the subject is being treated with an antidepressant. In some embodiments, the antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine. In some embodiments, the antidepressant is Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), PRAX-114, Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 3β-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05; Auvelity™), Carbidopa/oxitriptan (EVX-101), Cycloserine/lurasidone (NRX-101; Cyclurad), or Deudextromethorphan/quinidine (AVP-786, CTP-786).

In some embodiments, the method results in the attenuation of at least one clinical feature associated with a GI disease or condition. In some embodiments, the at least one clinical feature associated with a GI disease or condition is inflammation, jaundice, rectal bleeding, urgency, diarrhea, tenesmus, incontinence, fistula formation, constipation, bloating, abdominal cramps, colicky abdominal pain, change in bowel habit, mouth ulcers, anemia with associated symptoms of palpitations, dizziness, and dyspnea, fistulae, nausea, vomiting, fatigue, malaise, fever, or loss of weight or appetite.

In some embodiments, the method reduces the severity and/or duration of at least one symptom associated with the inflammatory disease or condition.

In some embodiments, the method further comprises administration of an additional therapeutic agent. In some embodiments, the additional therapeutic agent is an anti-inflammatory agent, an immunosuppressant drug, an 5-aminosalicylate (5-ASA), a corticosteroid, a tumor necrosis factor (TNF)-alpha inhibitor, an alpha-4 integrin inhibitor, an IL-12 and IL-23 inhibitor, a biologic, a biosimilar, an antibiotic, a dietary supplement, a laxative, an antispasmodic, an antidepressant, anti-diarrheal medication, a pain medication, or combinations thereof. In some embodiments, the additional therapeutic agent is adalimumab (Humira®), adalimumab-atto (Amjevita®), alosetron (Lotronex®), ampicillin (Omnipen®), azathioprine (Azasan®, Imuran®), balsalazide (Colazal®, Giazol®), cyclosporine (Gengraf®, Neoral®, Sandimmune®), certolizumab (Cimzia®), ciprofloxacin (Cipro®), eluxadoline (Viberzi®), golimumab (Simponi®), infliximab (Remicade®), infliximab-dyyb (Inflectra®), linaclotide (Linzess®), lubiprostone (Amitiza®), mesalamine (Asacol®, Canasa®, Delzicol®, Lialda®, Pentasa®), 6-mercaptopurine (Purinethol®, Purixan®), methotrexate (Trexall®, MTX®, Rheumatrex®, Mexate®), methylprednisolone, metronidazole (Flagyl®), natalizumab (Tysabri®), olsalazine (Dipentum®), prednisone, rifaximin (Xifaxan®), sulfasalazine (Azulfidine®), tacrolimus (Prograf®), tetracycline, ustekinumab (Stelara®), vedolizumab (Entyvio®), or combinations thereof.

In some embodiments, Compound (1) is administered. In some embodiments, a pharmaceutically acceptable salt of Compound (1) is administered.

In one aspect, the present disclosure is directed to a method of treating a gastrointestinal (GI) disease or condition in a subject in need thereof, comprising administering from about 20 mg to about 60 mg of Compound (1), or a pharmaceutically acceptable salt of Compound (1) at a dose equivalent of from about 20 mg to about 60 mg of the free base compound,

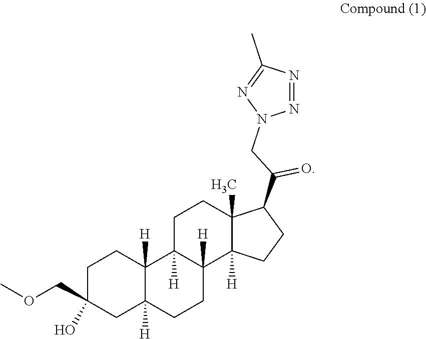

In one aspect, the present disclosure is directed to a method of treating irritable bowel syndrome (IBS) in a subject in need thereof, comprising administering a therapeutically effective amount of Compound (1), or a pharmaceutically acceptable salt thereof,

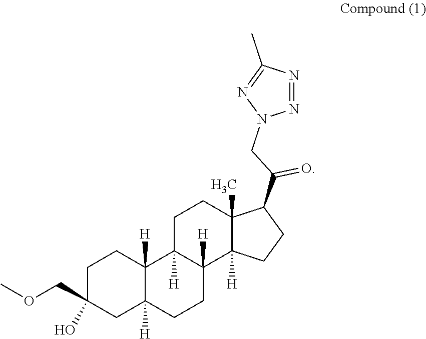

In one aspect, the present disclosure is directed to a method of treating irritable bowel syndrome (IBS) in a subject in need thereof, comprising administering from about 20 mg to about 60 mg of Compound (1), or a pharmaceutically acceptable salt of Compound (1) at a dose equivalent of from about 20 mg to about 60 mg of the free base compound,

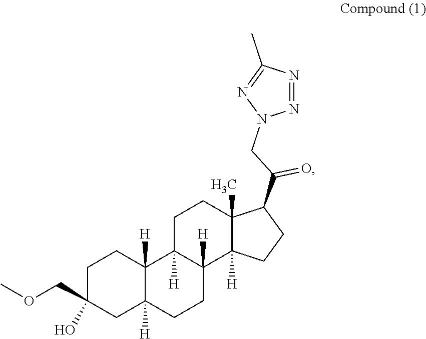

In one aspect, the present disclosure is directed to a method of treating a gastrointestinal (GI) disease or condition in a subject in need thereof, comprising administering a therapeutically effective amount of Compound (1), or a pharmaceutically acceptable salt thereof,

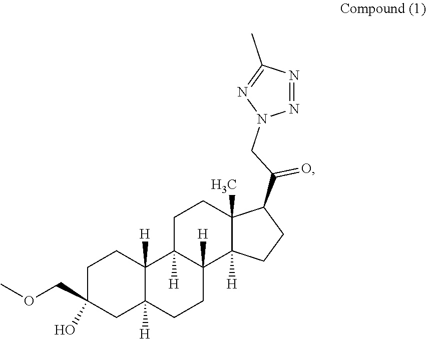

wherein the subject is being treated for a CNS-related disorder.

In one aspect, the present disclosure is directed to a method of treating irritable bowel syndrome (IBS) in a subject in need thereof, comprising administering a therapeutically effective amount of Compound (1), or a pharmaceutically acceptable salt thereof,

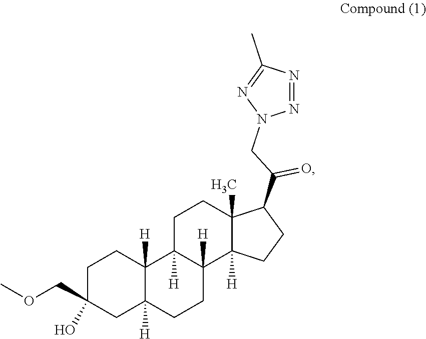

wherein the subject is being treated for a CNS-related disorder.

In one aspect, the present disclosure provides a method of treating a gastrointestinal (GI) disease or condition in a subject in need thereof, comprising administering a therapeutically effective amount of Compound (1):

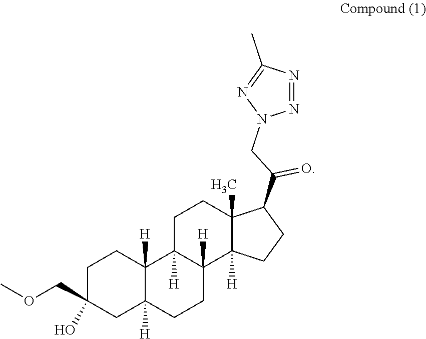

In one aspect, the present disclosure provides a method of treating a gastrointestinal (GI) disease or condition in a subject in need thereof, comprising administering a therapeutically effective amount of a pharmaceutically acceptable salt of Compound (1):

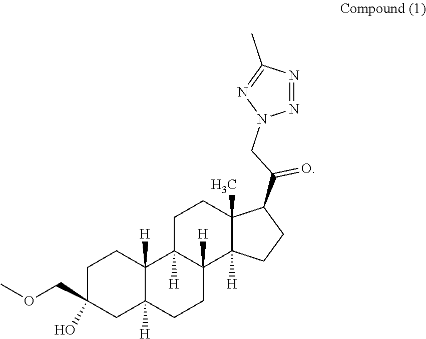

In some embodiments, the GI disease or condition is a non-inflammatory GI disease or condition. In some embodiments, the GI disease or condition is an inflammatory GI disease or condition.

In some embodiments, wherein the GI disease or condition is inflammatory bowel disease (IBD), ulcerative colitis (UC), Crohn's disease (CD), irritable bowel syndrome (IBS), indeterminate colitis (IC), microscopic colitis, collagenous colitis, lymphocytic colitis, incomplete microscopic colitis, segmental colitis associated with diverticula (SCAD), or combinations thereof.

In some embodiments, the GI disease or condition is irritable bowel syndrome (IBS). In some embodiments, the IBS is IBS with constipation (IBS-C), IBS with diarrhea (IBS-D), IBS with mixed constipation and diarrhea (IBS-M), IBS with alternating stool pattern (IBS-A), post-infection (PI) IBS, or post diverticulitis IBS.

In some embodiments, the subject has a CNS-related disorder.

In some embodiments, the subject is being treated for a CNS-related disorder.

In some embodiments, the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus. In some embodiments, the CNS-related disorder is a mood disorder. In some embodiments, the mood disorder is major depressive disorder or postpartum depression.

In some embodiments, the subject is being treated with an antidepressant. In some embodiments, the antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine. In some embodiments, the antidepressant is 4-Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), PRAX-114, Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 3β-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05; Auvelity™), Carbidopa/oxitriptan (EVX-101), Cycloserine/lurasidone (NRX-101; Cyclurad), or Deudextromethorphan/quinidine (AVP-786, CTP-786).

In some embodiments, the method results in the attenuation of at least one clinical feature associated with a GI disease or condition. In some embodiments, the at least one clinical feature associated with a GI disease or condition is inflammation, jaundice, rectal bleeding, urgency, diarrhea, tenesmus, incontinence, fistula formation, constipation, bloating, abdominal cramps, colicky abdominal pain, change in bowel habit, mouth ulcers, anemia with associated symptoms of palpitations, dizziness, and dyspnea, fistulae, nausea, vomiting, fatigue, malaise, fever, or loss of weight or appetite. In some embodiments, the method reduces the severity and/or duration of at least one symptom associated with the inflammatory disease or condition.

In some embodiments, the method further comprises administration of an additional therapeutic agent. In some embodiments, the additional therapeutic agent is an anti-inflammatory agent, an immunosuppressant drug, an 5-aminosalicylate (5-ASA), a corticosteroid, a tumor necrosis factor (TNF)-alpha inhibitor, an alpha-4 integrin inhibitor, an IL-12 and IL-23 inhibitor, a biologic, a biosimilar, an antibiotic, a dietary supplement, a laxative, an antispasmodic, an antidepressant, anti-diarrheal medication, a pain medication, or combinations thereof. In some embodiments, the additional therapeutic is adalimumab (Humira®), adalimumab-atto (Amjevita®), alosetron (Lotronex®), ampicillin (Omnipen®), azathioprine (Azasan®, Imuran®), balsalazide (Colazal®, Giazol®), cyclosporine (GengrafM, Neoral®, Sandimmune®), certolizumab (Cimzia®), ciprofloxacin (Cipro®), eluxadoline (Viberzi®), golimumab (Simponi®), infliximab (Remicade®), infliximab-dyyb (Inflectra®), linaclotide (Linzess®), lubiprostone (Amitiza®), mesalamine (Asacol®, Canasa®, Delzicol®, Lialda®, Pentasa®), 6-mercaptopurine (Purinethol®, Purixan®), methotrexate (Trexall®, MTX®, Rheumatrex®, Mexate®), methylprednisolone, metronidazole (Flagyl®), natalizumab (Tysabri®), olsalazine (Dipentum®), prednisone, rifaximin (Xifaxan®), sulfasalazine (Azulfidine®), tacrolimus (Prograf®), tetracycline, ustekinumab (Stelara®), vedolizumab (Entyvio®), or combinations thereof.

In some embodiments, Compound (1), or a pharmaceutically acceptable salt thereof, is administered once a day.

In some embodiments, Compound (1) is administered at a dose of about 10 mg to about 70 mg (e.g., about 15 mg to about 60 mg, about 25 mg to about 55 mg, or about 30 mg to about 50 mg). In some embodiments, Compound (1) is administered at a dose of about 15 mg, about 20 mg, about 25 mg, about 30 mg, about 35 mg, about 40 mg, about 45 mg, about 50 mg, about 55 mg, or about 60 mg. In some embodiments, Compound (1) is administered at a dose of about 10 mg to about 70 mg (e.g., about 15 mg to about 60 mg, about 25 mg to about 55 mg, or about 30 mg to about 50 mg) per dose per day. In some embodiments, Compound (1) is administered at a dose of about 15 mg, about 20 mg, about 25 mg, about 30 mg, about 35 mg, about 40 mg, about 45 mg, about 50 mg, about 55 mg, or about 60 mg per dose per day. In some embodiments, Compound (1) is administered at a dose of about 20 mg to about 60 mg. In some embodiments, Compound (1) is administered at a dose of about 50 mg. In some embodiments, Compound (1) is administered at a dose of about 60 mg. In some embodiments, Compound (1) is administered at a dose of about 50 mg per dose per day. In some embodiments, Compound (1) is administered at a dose of about 60 mg per dose per day.

In some embodiments, the pharmaceutically acceptable salt of Compound (1) is administered at a dose equivalent to about 10 mg to about 70 mg (e.g., about 15 mg to about 60 mg, about 25 mg to about 55 mg, or about 30 mg to about 50 mg). In some embodiments, the pharmaceutically acceptable salt of Compound (1) is administered at a dose equivalent to about 15 mg, about 20 mg, about 25 mg, about 30 mg, about 35 mg, about 40 mg, about 45 mg, about 50 mg, about 55 mg, or about 60 mg of the free base compound. In some embodiments, Compound (1) is administered at a dose of about 10 mg to about 70 mg (e.g., about 15 mg to about 60 mg, about 25 mg to about 55 mg, or about 30 mg to about 50 mg) per dose per day. In some embodiments, Compound (1) is administered at a dose of about 15 mg, about 20 mg, about 25 mg, about 30 mg, about 35 mg, about 40 mg, about 45 mg, about 50 mg, about 55 mg, or about 60 mg per dose per day. In some embodiments, the pharmaceutically acceptable salt of Compound (1) is administered at a dose equivalent to about 50 mg of the free base compound. In some embodiments, the pharmaceutically acceptable salt of Compound (1) is administered at a dose equivalent to about 60 mg of the free base compound. In some embodiments, the pharmaceutically acceptable salt of Compound (1) is administered at a dose equivalent to about 50 mg of the free base compound per dose per day. In some embodiments, the pharmaceutically acceptable salt of Compound (1) is administered at a dose equivalent to about 60 mg of the free base compound per dose per day.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasally, or transdermally. In some embodiments, Compound (1), or the pharmaceutically acceptable salt of Compound (1), is administered orally.

With oral dosing, one to five and especially two to four and typically three oral doses per day are representative regimens. Using these dosing patterns, each dose provides from about 0.01 to about 20 mg/kg (e.g., from about 0.1 to about 10 mg/kg, from about 0.2 to about 5 mg/kg, from about 0.1 to about 1 mg/kg, from about 0.2 to about 0.8 mg/kg, from about 0.2 to about 0.7 mg/kg, or from about 0.2 to about 0.5 mg/kg) of Compound (1), or the pharmaceutically acceptable salt of Compound (1), provided herein.

In one aspect, the present disclosure provides a method of treating a gastrointestinal (GI) disease or condition in a subject in need thereof, comprising administering about 20 mg to about 60 mg of Compound (1):

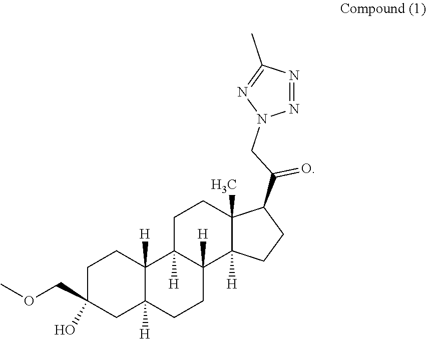

In one aspect, the present disclosure provides a method of treating a gastrointestinal (GI) disease or condition in a subject in need thereof, comprising administering a pharmaceutically acceptable salt of Compound (1) at a dose equivalent to about 20 mg to about 60 mg of the free base compound:

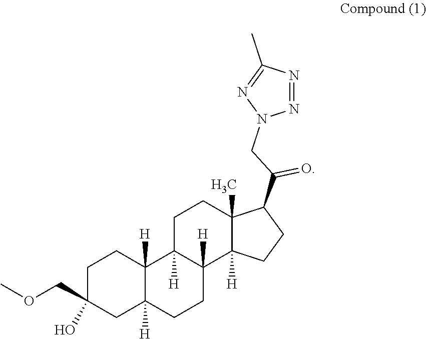

In one aspect, the present disclosure provides a method of treating irritable bowel syndrome (IBS) in a subject in need thereof, comprising administering a therapeutically effective amount of Compound (1):

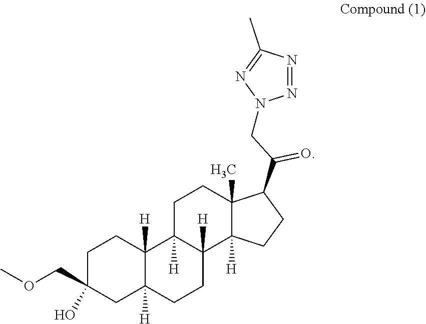

In one aspect, the present disclosure provides a method of treating irritable bowel syndrome (IBS) in a subject in need thereof, comprising administering a therapeutically effective amount of a pharmaceutically acceptable salt of Compound (1):

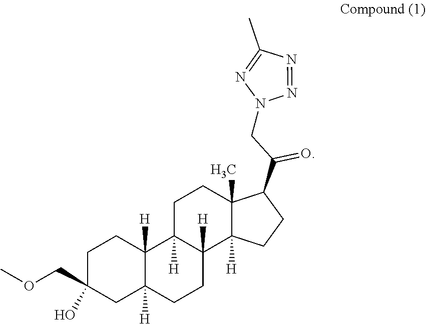

In one aspect, the present disclosure provides a method of treating irritable bowel syndrome (IBS) in a subject in need thereof, comprising administering about 20 mg to about 60 mg of Compound (1):

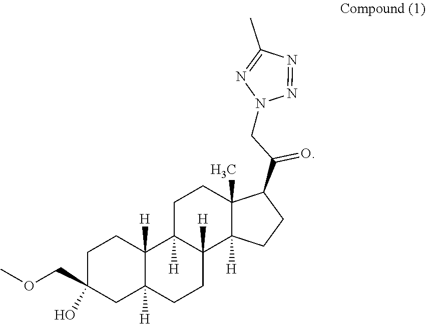

In one aspect, the present disclosure provides a method of treating irritable bowel syndrome (IBS) in a subject in need thereof, comprising administering a pharmaceutically acceptable salt of Compound (1) at a dose equivalent to about 20 mg to about 60 mg of the free base compound:

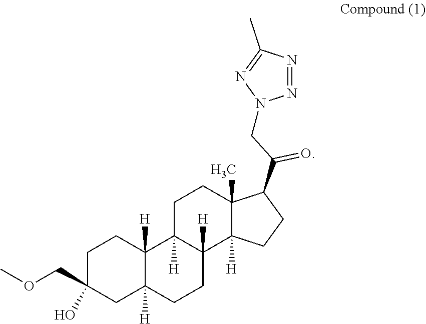

In one aspect, the present disclosure provides a method of treating a gastrointestinal (GI) disease or condition in a subject in need thereof, comprising administering a therapeutically effective amount of Compound (1):

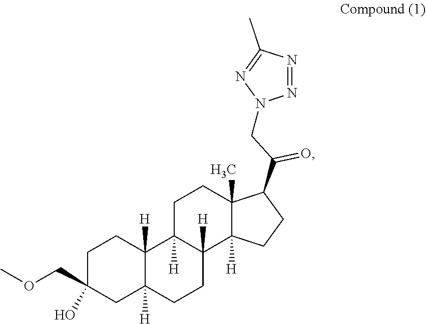

wherein the subject is being treated for a CNS-related disorder.

In one aspect, the present disclosure provides a method of treating a gastrointestinal (GI) disease or condition in a subject in need thereof, comprising administering a therapeutically effective amount of a pharmaceutically acceptable salt of Compound (1):

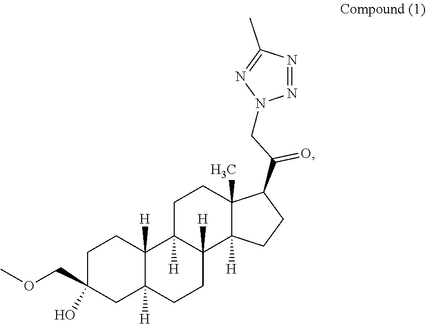

wherein the subject is being treated for a CNS-related disorder.

In one aspect, the present disclosure provides a method of treating irritable bowel syndrome (IBS) in a subject in need thereof, comprising administering a therapeutically effective amount of Compound (1):

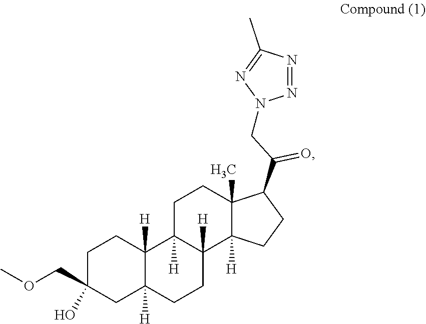

wherein the subject is being treated for a CNS-related disorder.

In one aspect, the present disclosure provides a method of treating irritable bowel syndrome (IBS) in a subject in need thereof, comprising administering a therapeutically effective amount of a pharmaceutically acceptable salt of Compound (1):

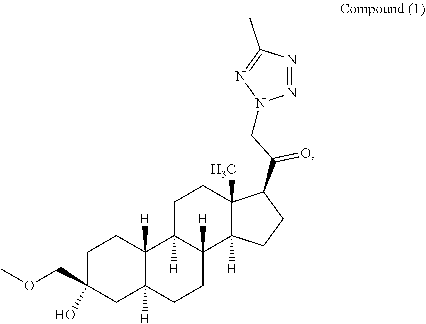

wherein the subject is being treated for a CNS-related disorder.

### DETAILED DESCRIPTION

### Definitions

As used herein, “Compound (1)” refers to the compound having the formula (or structure):

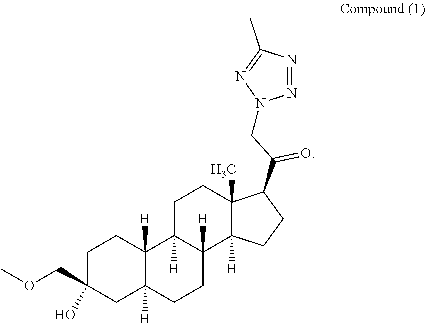

Compound (1) is also known as 3alpha-hydroxy-3beta-methoxymethyl-21-(5-methyl-2H-tetrazol-2-yl)-19-nor-5 alpha-pregnan-20-one, and by its IUPAC name: 1-((3R,5S,8R,9R,10S,13S,14S,17S)-3-hydroxy-3-(methoxymethyl)-13-methylhexadecahydro-1H-cyclopenta[a]phenanthren-17-yl)-2-(5-methyl-2H-tetrazol-2-yl)ethan-1-one (CAS Registry Number. 1832596-09-4). A method of chemically synthesizing Compound (1), was described in PCT Application Publication No. WO 2015/180679, which is incorporated by reference in its entirety herein.

Compound (1) is a neuroactive steroids that has been shown to be a positive allosteric modulator of GABA_{A} receptors that target synaptic and extrasynaptic GABA_{A} receptors. As a positive allosteric modulator of GABA_{A} receptors, Compound (1) serves as therapeutic agent to treat CNS related disorders, e.g., depression, postpartum depression and major depressive disorder and to treat neurological conditions, e.g., essential tremor, epilepsy, and Parkinson's disease.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

The chemical elements are identified in accordance with the Periodic Table of the Elements, CAS version, Handbook of Chemistry and Physics, 75ili Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5ili Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

As used herein, the term “about”, when referring to a numerical value or range, allows for a degree of variability in the value or range, for example, within 10%, or within 5% of a stated value or of a stated limit of a range.

As used herein, the term “modulation” refers to the inhibition or potentiation of GABA_{A}receptor function. A “modulator” (e.g., a compound or pharmaceutically acceptable salt thereof that modulates GABA_{A} receptor function) may be, for example, an agonist, partial agonist, antagonist, or partial antagonist of the GABA_{A} receptor.

As used herein, and unless otherwise specified, the terms “treat,” “treating” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

The terms “disease”, “disorder”, and “condition” are used interchangeably herein.

A “GI disease” or “condition” is a disease or condition related to the gastrointestinal tract, e.g., the esophagus, stomach, small intestine, large intestine, rectum, and the accessory organs of digestion, including the liver, gallbladder, and pancreas.

As used herein, an “effective amount” of a compound (or pharmaceutically acceptable salt thereof) refers to an amount sufficient to elicit the desired biological response, e.g., to treat a gastrointestinal disease or condition, e.g., to treat irritable bowel syndrome. As will be appreciated by those of ordinary skill in this art, the effective amount of a compound (or pharmaceutically acceptable salt thereof) of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound (or pharmaceutically acceptable salt thereof) is an amount sufficient to provide a therapeutic benefit in the treatment of a disease, disorder or condition, or to delay or minimize one or more symptoms associated with the disease, disorder or condition. A therapeutically effective amount of a compound (or pharmaceutically acceptable salt thereof) means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

A “subject” or “patient” is a human (e.g., a male or female of any age group, e.g., a pediatric subject (e.g., infant, child, adolescent) or adult subject (e.g., young adult, middle-aged adult or senior adult)).

As used herein, the term “dose equivalent” means a bioequivalent dose. For example, the dose equivalent of a pharmaceutically acceptable salt of Compound (1) for a 50 mg dose of Compound (1) is the amount of the pharmaceutically acceptable salt (by weight) needed to provide a bioequivalent dose to the 50 mg dose of the free base of Compound (1).

As used herein, the term “unit dosage form” is defined to refer to the form in which Compound (1) is administered to the patient. Specifically, the unit dosage form can be, for example, a pill, capsule, or tablet. In some embodiments, the unit dosage form is a capsule. In some embodiments, the unit dosage form is a tablet.

As used herein, “solid dosage form” means a pharmaceutical dose(s) in solid form, e.g., tablets, capsules, granules, powders, sachets, reconstitutable powders, dry powder inhalers and chewables.

As used herein, “ameliorate,” “amelioration,” “improvement” or the like refers to, for example, a detectable improvement or a detectable change consistent with improvement that occurs in a subject. Such improvement or change may be observed in treated subjects compared to subjects not treated, e.g., with Compound (1) or a pharmaceutically acceptable salt thereof.

Amelioration of a disease, condition, or symptom may be determined subjectively or objectively, e.g., self-assessment by a subject, by a clinician's assessment or by conducting an appropriate assay or measurement. Amelioration may be transient, prolonged, or permanent, or it may be variable at relevant times during or after treatment.

As used herein, the terms “administer,” “administering,” or “administration” refer to implanting, absorbing, ingesting, injecting, inhaling, or otherwise introducing a compound described herein, or a composition thereof, in or on a subject.

### Methods of Treatment

The present disclosure is directed to methods of treating gastrointestinal (GI) diseases or conditions.

Gastrointestinal treatment-emergent adverse events (TEAEs), are frequently observed in patients with major depressive disorder (MDD) while taking antidepressants and may lead to treatment discontinuation. For example, one study (Oliva V. et al. Prog Neuropsychopharmacol Biol Psychiatry. 2021 Jul. 13; 109:110266) found that escitalopram and sertraline were the least tolerated antidepressants on the gastrointestinal tract, being associated with all the considered TEAEs (nausea/vomiting, diarrhea, constipation, abdominal pain, dyspepsia, anorexia, increased appetite and dry mouth) with the exception of constipation and increased appetite.

Drugs with effects on serotonin receptors or serotonin levels can affect gastric motility (Janssen P, Vos R, Tack J: The influence of citalopram on interdigestive gastrointestinal motility in man. Aliment Pharmacol Ther 2010; 32:289-295). Also, serotonergic compounds acting on central 5-HT₃ receptors can lead to nausea and vomiting (Browning K N: Role of central vagal 5-HT3 receptors in gastrointestinal physiology and pathophysiology. Front Neurosci 2015; 9:413). Some of the most frequently reported side effects associated with the use of SSRIs include nausea, diarrhea, dyspepsia, GI bleeding, and abdominal pain. (Goldstein B J, Goodnick P J: Selective serotonin reuptake inhibitors in the treatment of affective disorders—III: tolerability, safety and pharmacoeconomics. J Psychopharmacol 1998; 12:S55-S87; Uher R, et al. Adverse reactions to antidepressants. Br J Psychiatry 2009; 195:202-210). Some studies have found nausea and vomiting to be one of the most common reasons for treatment discontinuation.

A meta-analysis of adverse reactions reported during clinical trials indicated that when compared to SSRIs and duloxetine, venlafaxine had higher rates of nausea and vomiting, while sertraline appeared to be have a higher incidence of diarrhea when compared to other SSRIs and venlafaxine (Gartlehner G, et al. Drug Class Review: Second-Generation Antidepressants: Final Update 5 Report. Drug Class Reviews. Portland, Oregon Health and Science University, 2011). Another meta-analysis found that all 15 of the most commonly used antidepressants in MDD (agomelatine, bupropion, citalopram, desvenlafaxine, duloxetine, escitalopram, fluoxetine, fluvoxamine, levomilnacipran, mirtazapine, paroxetine, reboxetine, sertraline, venlafaxine, and vortioxetine) showed higher rates of gastrointestinal adverse reactions compared to placebo. Escitalopram and sertraline were the least tolerated antidepressants on the GI tract, while mirtazapine was the antidepressant with fewer side effects on the gut, being only associated with increased appetite (Oliva V, et al. Gastrointestinal side effects associated with antidepressant treatments in patients with major depressive disorder: A systematic review and meta-analysis. Prog Neuropsychopharmacol Biol Psychiatry. 2021 Jul. 13; 109:110266).

In one aspect, the present disclosure provides a method of method of treating a gastrointestinal (GI) disease or condition in a subject in need thereof, comprising administering a therapeutically effective amount of Compound (1), or a pharmaceutically acceptable salt thereof,

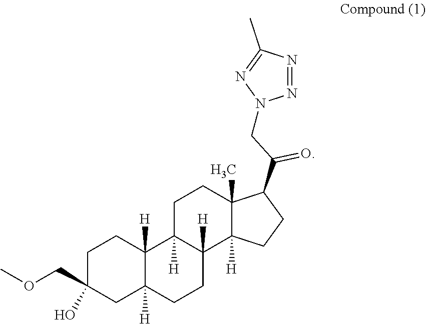

In some embodiments, the GI disease or condition is a non-inflammatory GI disease or condition. In some embodiments, the GI disease or condition is an inflammatory GI disease or condition.

In some embodiments, the GI disease or condition is inflammatory bowel disease (IBD), ulcerative colitis (UC), Crohn's disease (CD), irritable bowel syndrome (IBS), indeterminate colitis (IC), microscopic colitis, collagenous colitis, lymphocytic colitis, incomplete microscopic colitis, segmental colitis associated with diverticula (SCAD), or combinations thereof. In some embodiments, the GI disease or condition is irritable bowel syndrome (IBS). In some embodiments, the IBS is IBS with constipation (IBS-C), IBS with diarrhea (IBS-D), IBS with mixed constipation and diarrhea (IBS-M), IBS with alternating stool pattern (IBS-A), post-infection (PI) IBS, or post diverticulitis IBS.

In some embodiments, the subject has a CNS-related disorder and/or the subject is being treated for a CNS-related disorder. In some embodiments, the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus. In some embodiments, the CNS-related disorder is a mood disorder. In some embodiments, the mood disorder is major depressive disorder or postpartum depression.

In some embodiments, the subject is being treated with an antidepressant. In some embodiments, the antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine. In some embodiments, the antidepressant is Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), PRAX-114, Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 3β-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05; Auvelity™), Carbidopa/oxitriptan (EVX-101), Cycloserine/lurasidone (NRX-101; Cyclurad), or Deudextromethorphan/quinidine (AVP-786, CTP-786).

In some embodiments, the method results in the attenuation of at least one clinical feature associated with a GI disease or condition. In some embodiments, the at least one clinical feature associated with a GI disease or condition is inflammation, jaundice, rectal bleeding, urgency, diarrhea, tenesmus, incontinence, fistula formation, constipation, bloating, abdominal cramps, colicky abdominal pain, change in bowel habit, mouth ulcers, anemia with associated symptoms of palpitations, dizziness, and dyspnea, fistulae, nausea, vomiting, fatigue, malaise, fever, or loss of weight or appetite.

In some embodiments, the method reduces the severity and/or duration of at least one symptom associated with the inflammatory disease or condition.

In some embodiments, the method further comprises administration of an additional therapeutic agent. In some embodiments, the additional therapeutic agent is an anti-inflammatory agent, an immunosuppressant drug, an 5-aminosalicylate (5-ASA), a corticosteroid, a tumor necrosis factor (TNF)-alpha inhibitor, an alpha-4 integrin inhibitor, an IL-12 and IL-23 inhibitor, a biologic, a biosimilar, an antibiotic, a dietary supplement, a laxative, an antispasmodic, an antidepressant, anti-diarrheal medication, a pain medication, or combinations thereof. In some embodiments, the additional therapeutic agent is adalimumab (Humira®), adalimumab-atto (Amjevita®), alosetron (Lotronex®), ampicillin (Omnipen®), azathioprine (Azasan®, Imuran®), balsalazide (Colazal®, Giazol®), cyclosporine (GengrafM, Neoral®, Sandimmune®), certolizumab (Cimzia®), ciprofloxacin (Cipro®), eluxadoline (Viberzi®), golimumab (Simponi®), infliximab (Remicade®), infliximab-dyyb (Inflectra®), linaclotide (Linzess®), lubiprostone (Amitiza®), mesalamine (Asacol®, Canasa®, Delzicol®, Lialda®, Pentasa®), 6-mercaptopurine (Purinethol®, Purixan®), methotrexate (Trexall®, MTX®, Rheumatrex®, Mexate®), methylprednisolone, metronidazole (Flagyl®), natalizumab (Tysabri®), olsalazine (Dipentum®), prednisone, rifaximin (Xifaxan®), sulfasalazine (Azulfidine®), tacrolimus (Prograf®), tetracycline, ustekinumab (Stelara®), vedolizumab (Entyvio®), or combinations thereof.

In some embodiments, Compound (1) is administered. In some embodiments, a pharmaceutically acceptable salt of Compound (1) is administered.

In one aspect, the present disclosure is directed to a method of treating a gastrointestinal (GI) disease or condition in a subject in need thereof, comprising administering from about 20 mg to about 60 mg of Compound (1), or a pharmaceutically acceptable salt of Compound (1) at a dose equivalent of from about 20 mg to about 60 mg of the free base compound,

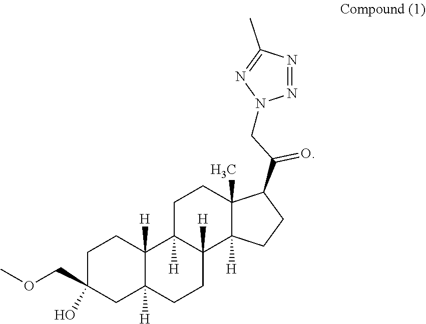

In one aspect, the present disclosure is directed to a method of treating irritable bowel syndrome (IBS) in a subject in need thereof, comprising administering a therapeutically effective amount of Compound (1), or a pharmaceutically acceptable salt thereof,

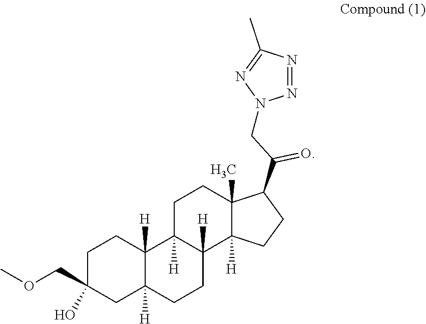

In one aspect, the present disclosure is directed to a method of treating irritable bowel syndrome (IBS) in a subject in need thereof, comprising administering from about 20 mg to about 60 mg of Compound (1), or a pharmaceutically acceptable salt of Compound (1) at a dose equivalent of from about 20 mg to about 60 mg of the free base compound,

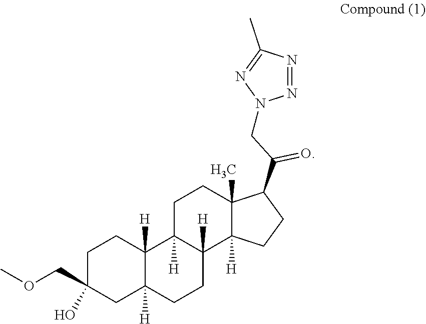

In one aspect, the present disclosure is directed to a method of treating a gastrointestinal (GI) disease or condition in a subject in need thereof, comprising administering a therapeutically effective amount of Compound (1), or a pharmaceutically acceptable salt thereof,

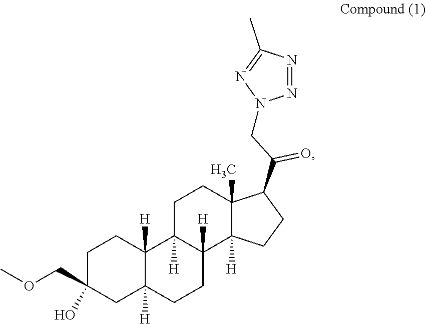

wherein the subject is being treated for a CNS-related disorder.

In one aspect, the present disclosure is directed to a method of treating irritable bowel syndrome (IBS) in a subject in need thereof, comprising administering a therapeutically effective amount of Compound (1), or a pharmaceutically acceptable salt thereof,

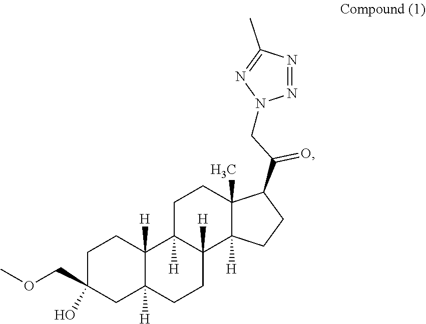

wherein the subject is being treated for a CNS-related disorder.

In one aspect, the present disclosure is directed to a method of treating a gastrointestinal (GI) disease or condition in a subject in need thereof, comprising administering a therapeutically effective amount of Compound (1):

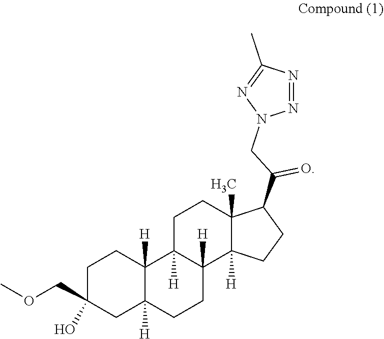

In one aspect, the present disclosure is directed to a method of treating a gastrointestinal (GI) disease or condition in a subject in need thereof, comprising administering a therapeutically effective amount of a pharmaceutically acceptable salt of Compound (1):

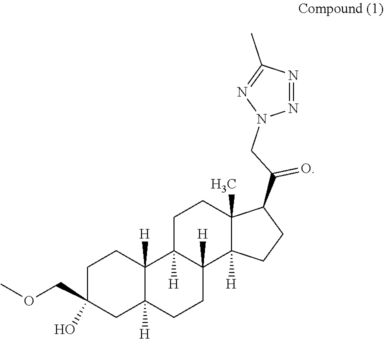

In some embodiments, the GI disease or condition is a non-inflammatory GI disease or condition.

In some embodiments, the GI disease or condition is an inflammatory GI disease or condition.

Gastrointestinal diseases and conditions include, but are not limited to, constipation, diarrhea, gas-bloat syndrome, enteritis, esophagitis, hemorrhoids, anal fissures, perianal abscesses, anal fistulas, perianal infections, diverticular diseases, colitis, colon polyps, ulcers, dyspepsia, acute gastrointestinal bleeding, lower esophageal mucosal rings, esophageal strictures, esophageal dismotility, hiatal hernia, achalasia, irritable bowel syndrome (IBS), Barrett's esophagus, gastroparesis, gastrointestinal motility disorders, celiac disease, IgA deficiency related GI disease, diverticulosis, diverticulitis, malabsorption syndromes, gastroesophageal reflux disease (GERD), problems caused by gastric bypass surgery, belching, eructation, flatulence, diarrhea, inflammatory bowel disease (IBD), ulcerative colitis (UC), Crohn's disease (CD), an indeterminate colitis (IC), a microscopic colitis, a collagenous colitis, a lymphocytic colitis, an incomplete microscopic colitis, and a segmental colitis associated with diverticula (SCAD), infectious enteritis, idiopathic gastric acid hypersecretion, gastritis, constipation, colic, vomiting, nausea, cyclic vomiting syndrome, cannabinoid hyperemesis syndrome, motion sickness, gastrointestinal injuries, esophageal injuries, gastric mucosal injuries, short bowel syndrome, bowel dysfunctions, early satiety, abdominal pain, abdominal bloating, sour stomach, radiation-induced injury to the gastrointestinal tract, gastrointestinal diseases or conditions induced by medications, chronic sore throat, noncardiac chest pains, coughing, dysphagia, Shwachman syndrome, decreased gastric mucin production, iron deficiency anemia, cystic fibrosis, cancer, pancreatitis, cholangitis, cholecystitis, cirrhosis, and the like.

In some embodiments, the GI disease or condition is inflammatory bowel disease (IBD), ulcerative colitis (UC), Crohn's disease (CD), irritable bowel syndrome (IBS), indeterminate colitis (IC), microscopic colitis, collagenous colitis, lymphocytic colitis, incomplete microscopic colitis, segmental colitis associated with diverticula (SCAD), or combinations thereof.

In some embodiments, the GI disease or condition is irritable bowel syndrome (IBS). IBS can include a group of functional bowel diseases or conditions characterized by one or more symptoms including, but not limited to, abdominal pain, abdominal discomfort, change in bowel pattern, loose or more frequent bowel movements, diarrhea, and constipation, typically in the absence of any apparent structural abnormality. There are at least three forms of IBS, depending on which symptom predominates: (1) diarrhea-predominant (IBS-D); (2) constipation-predominant (IBS-C); and (3) IBS with alternating stool pattern (IBS-A). IBS can also occur in the form of a mixture of symptoms (IBS-M). There are also various clinical subtypes of IBS, such as post-infectious IBS (IBS-PI) and a post-diverticulitis IBS. In some embodiments, IBS can be associated with inflammation. In some embodiments, IBS is associated with inflammation in subjects with diarrhea-predominant (IBS-D) or with presumed post-infectious IBS (IBS-PI). When IBS is associated with inflammation, mucosal immune system activation may be characterized by alterations in particular immune cells and markers. For example, in some embodiments, an increased numbers of lymphocytes may be present in the colon and/or small intestine of a subject with IBS. In some embodiments, an increased number of mast cells may be present in the terminal ileum, jejunum, and/or colon of a subject with IBS. In some embodiments, elevated levels of plasma pro-inflammatory interleukins may be present in subjects with IBS. In other embodiments, IBS is not associated with inflammation. In some embodiments, the IBS is IBS with constipation (IBS-C), IBS with diarrhea (IBS-D), IBS with mixed constipation and diarrhea (IBS-M), IBS with alternating stool pattern (IBS-A), post-infection (PI) IBS, or post diverticulitis IBS.

In some embodiments, the GI disease or condition to be treated may be one associated with inflammation. In some embodiments, the disease may be a condition that results from, is exacerbated by, or is otherwise related to inflammation. In some embodiments, the disease may be a condition that may be treated or alleviated by reducing inflammation.

In some embodiments, the method results in the attenuation of at least one clinical feature associated with a GI disease or condition. In some embodiments, the at least one clinical feature associated with a GI disease or condition is inflammation, jaundice, rectal bleeding, urgency, diarrhea, tenesmus, incontinence, fistula formation, constipation, bloating, abdominal cramps, colicky abdominal pain, change in bowel habit, mouth ulcers, anemia with associated symptoms of palpitations, dizziness, and dyspnea, fistulae, nausea, vomiting, fatigue, malaise, fever, or loss of weight or appetite.

In some embodiments, the method reduces the severity and/or duration of at least one symptom associated with the inflammatory disease or condition.

In some embodiments, the methods described herein reduce the severity, frequency, and/or duration of at least one symptom associated with a GI disease or condition, optionally, wherein the methods induce remission of a GI disease or condition. Several methods, as known in the art, can be used to determine if a subject having a GI disease or condition responds to a treatment, e.g., as described herein. These methods can also be used to determine whether a method described herein induces remission of the GI disease or condition.

For example, in some embodiments, the Rome IV Criteria can be used to determine if a patient responds to treatment. The Rome IV Criteria or Rome Criteria is a research tool used to evaluate a patient with irritable bowel syndrome (Fass R, et al. Archives of Internal Medicine. 161(17): 2081-8, 2001; Talley NJ. Reviews in Gastroenterological Disorders. 6 (2): 72-82, 2006). The Rome IV criteria includes recurrent abdominal pain, on average, at least 1 day/week in the last 3 months, associated with two or more of the following criteria: (i) related to defecation, (ii) associated with a change in frequency of stool, and (iii) associated with a change in form (appearance) of stool.

In some embodiments, the subject to be treated with the methods described herein has a CNS-related disorder. In some embodiments, the subject to be treated with the methods described herein is being treated for a CNS-related disorder.

Exemplary CNS-related disorders include, but are not limited to, sleep disorders [e.g., insomnia], mood disorders [e.g., depression (e.g., major depressive disorder (MDD) or postpartum depression (PPD)), dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD))], schizophrenia spectrum disorders [e.g., schizophrenia, schizoaffective disorder], convulsive disorders [e.g., epilepsy (e.g., status epilepticus (SE)), seizures], disorders of memory and/or cognition [e.g., attention disorders (e.g., attention deficit hyperactivity disorder (ADHD)), dementia (e.g., Alzheimer's type dementia, Lewis body type dementia, vascular type dementia], movement disorders [e.g., Huntington's disease, Parkinson's disease], personality disorders [e.g., anti-social personality disorder, obsessive compulsive personality disorder], autism spectrum disorders (ASD) [e.g., autism, monogenetic causes of autism such as synaptophathy's, e.g., Rett syndrome, Fragile X syndrome, Angelman syndrome], pain [e.g., neuropathic pain, injury related pain syndromes, acute pain, chronic pain], traumatic brain injury (TBI), vascular diseases [e.g., stroke, ischemia, vascular malformations], substance abuse disorders and/or withdrawal syndromes [e.g., addition to opiates, cocaine, and/or alcohol], and tinnitus.

In some embodiments, the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus.

In some embodiments, the CNS-related disorder is a mood disorder. In some embodiments, the mood disorder is major depressive disorder. In some embodiments, the mood disorder is postpartum depression. In some embodiments, the mood disorder is major depressive disorder or postpartum depression.

In some embodiments, the subject is being treated with an antidepressant. In some embodiments, the subject experiences a GI disease or condition as a result of treatment with the antidepressant.

In some embodiments, the antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

In some embodiments, the antidepressant is 4-Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), PRAX-114, Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 30-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05; Auvelity™), Carbidopa/oxitriptan (EVX-101), Cycloserine/lurasidone (NRX-101; Cyclurad), or Deudextromethorphan/quinidine (AVP-786, CTP-786).

In some embodiments, the antidepressant is an SSRI. In some embodiments, the SSRI is sertraline, escitalopram, citalopram, fluvoxamine, paroxetine, fluoxetine, indalpine, or zimelidine. In some embodiments, the SSRI is sertraline. In some embodiments, the SSRI is escitalopram. In some embodiments, the SSRI is citalopram.

In some embodiments, the antidepressant is an SNRI. In some embodiments, the SNRI is duloxetine, desvenlafaxine, levomilnacipran, milnacipran, or venlafaxine. In some embodiments, the SNRI is duloxetine. In some embodiments, the SNRI is desvenlafaxine.

In some embodiments, the antidepressant is an SMS. In some embodiments, the SMS is vilazodone or vortioxetine.

In some embodiments, the antidepressant is an SARI. In some embodiments, the SARI is nefazodone, trazodone, or etoperidone.

In some embodiments, the antidepressant is an NRI. In some embodiments, the NRI is reboxetine, teniloxazine, viloxazine, or atomoxetine.

In some embodiments, the antidepressant is a NDRI. In some embodiments, the NDRI is bupropion, amphetamine, methylphenidate, modafinil, amineptine, or nomifensine.

In some embodiments, the antidepressant is a TCA. In some embodiments, the TCA is amitriptyline, amitriptylinoxide, clomipramine, desipramine, diebnzepin, dimetacrine, dosluepin, doxepin, imipramine, lofepramine, melitracen, nitroxazepine, notriptyline, noxiptiline, opipramol, pipofezine, protriptyline, trimipramine, butriptyline, demexiptiline, fluacizine, imipraminoxide, iprindole, metapramine, propizepine, quinupramine, tiazesim, or tofenacin.

In some embodiments, the antidepressant is a TeCA. In some embodiments, the TeCA is amoxapine, maprotiline, mianserin, mirtazapine, or setiptiline.

In some embodiments, the antidepressant is a MAOI. In some embodiments, the MAOI is isocarboxazid, phenelzine, tranylcypromine, benmoxin, iproclozide, iproniazid, mebanazine, nialamide, octamoxin, pheniprazine, phenoxypropazine, pivhydrazine, safrazine, selegiline, caroxazone, metralindole, moclobemide, pirlindole, eprobemide, minaprine, toloxatone, or bifemelane.

In some embodiments, the antidepressant is an atypical antipsychotic. In some embodiments, the atypical antipsychotic is amisulpride, lumateperone, lurasidone, quetiapine, aripiprazole, brexpiprazole, lumateperone, lurasidone, olanzapine, quetiapine, or risperidone.

In some embodiments, the antidepressant is agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

In some embodiments, the antidepressant is administered per its labeled prescribing information.

### Additional Therapeutic Agent

In some embodiments, the methods described herein further comprise administration of an additional therapeutic agent.

In some embodiments, the additional therapeutic agent is an anti-inflammatory agent, an immunosuppressant drug, an 5-aminosalicylate (5-ASA), a corticosteroid, a tumor necrosis factor (TNF)-alpha inhibitor, an alpha-4 integrin inhibitor, an IL-12 and IL-23 inhibitor, a biologic, a biosimilar, an antibiotic, a dietary supplement, a laxative, an antispasmodic, an antidepressant, anti-diarrheal medication, a pain medication, or combinations thereof.

In some embodiments, the additional therapeutic is adalimumab (Humira®), adalimumab-atto (Amjevita®), alosetron (Lotronex®), ampicillin (Omnipen®), azathioprine (Azasan®, Imuran®), balsalazide (Colazal®, Giazol®), cyclosporine (GengrafM, Neoral®, Sandimmune®), certolizumab (Cimzia®), ciprofloxacin (Cipro®), eluxadoline (Viberzi®), golimumab (Simponi®), infliximab (Remicade®), infliximab-dyyb (Inflectra®), linaclotide (Linzess®), lubiprostone (Amitiza®), mesalamine (Asacol®, Canasa®, Delzicol®, Lialda®, Pentasa®), 6-mercaptopurine (Purinethol®, Purixan®), methotrexate (Trexall®, MTX®, Rheumatrex®, Mexate®), methylprednisolone, metronidazole (Flagyl®), natalizumab (Tysabri®), olsalazine (Dipentum®), prednisone, rifaximin (Xifaxan®), sulfasalazine (Azulfidine®), tacrolimus (Prograf®), tetracycline, ustekinumab (Stelara®), vedolizumab (Entyvio®), or combinations thereof.

In some embodiments, the administration of Compound (1), or a pharmaceutically acceptable salt thereof, and the additional therapeutic agent can proceed by any technique apparent to those of skill in the art including, for example, separate, sequential, concurrent and alternating administration.

In some embodiments, Compound (1), or a pharmaceutically acceptable salt thereof, and the additional therapeutic agent are administered in separate dosage forms. In some embodiments, Compound (1), or a pharmaceutically acceptable salt thereof, and the additional therapeutic agent are administered at the same time. In some embodiments, Compound (1), or a pharmaceutically acceptable salt thereof, and the additional therapeutic agent are administered at different times.

In some embodiments, the additional therapeutic agent can be administered simultaneously with Compound (1), or a pharmaceutically acceptable salt thereof. In some embodiments, the additional therapeutic agent and Compound (1), or a pharmaceutically acceptable salt thereof, can be administered sequentially.

In one aspect, the present disclosure provides a method of treating a gastrointestinal (GI) disease or condition in a subject in need thereof, comprising administering about 20 mg to about 60 mg of Compound (1):

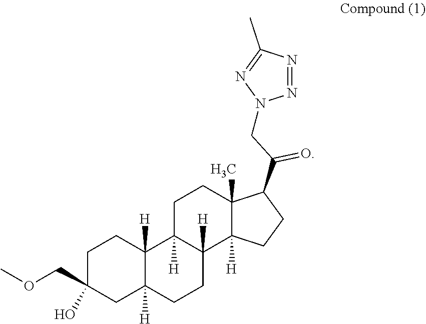

In one aspect, the present disclosure provides a method of treating a gastrointestinal (GI) disease or condition in a subject in need thereof, comprising administering a pharmaceutically acceptable salt of Compound (1) at a dose equivalent to about 20 mg to about 60 mg of the free base compound:

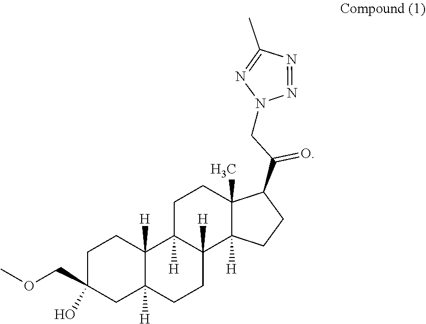

In one aspect, the present disclosure provides a method of treating irritable bowel syndrome (IBS) in a subject in need thereof, comprising administering a therapeutically effective amount of Compound (1):

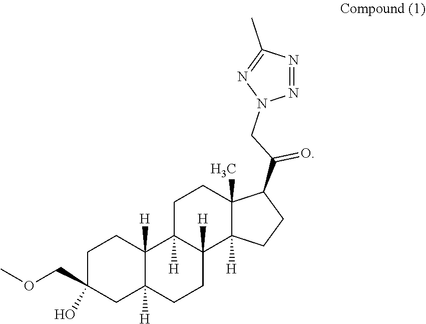

In one aspect, the present disclosure provides a method of treating irritable bowel syndrome (IBS) in a subject in need thereof, comprising administering a therapeutically effective amount of a pharmaceutically acceptable salt of Compound (1):

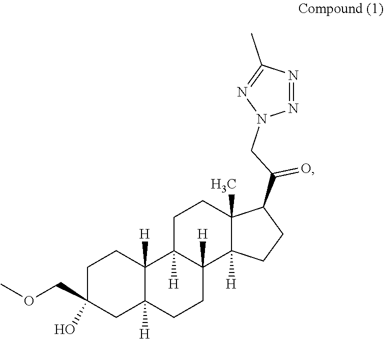

In one aspect, the present disclosure provides a method of treating irritable bowel syndrome (IBS) in a subject in need thereof, comprising administering about 20 mg to about 60 mg of Compound (1):

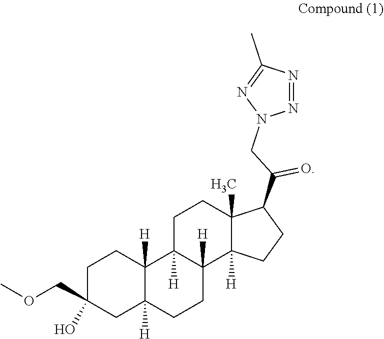

In one aspect, the present disclosure provides a method of treating irritable bowel syndrome (IBS) in a subject in need thereof, comprising administering a pharmaceutically acceptable salt of Compound (1) at a dose equivalent to about 20 mg to about 60 mg of the free base compound:

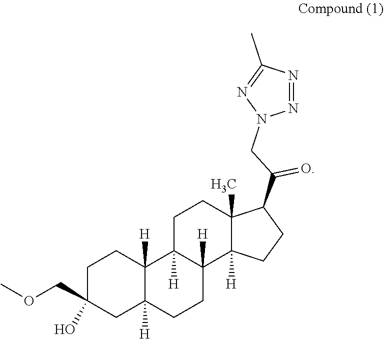

In one aspect, the present disclosure provides a method of treating a gastrointestinal (GI) disease or condition in a subject in need thereof, comprising administering a therapeutically effective amount of Compound (1):

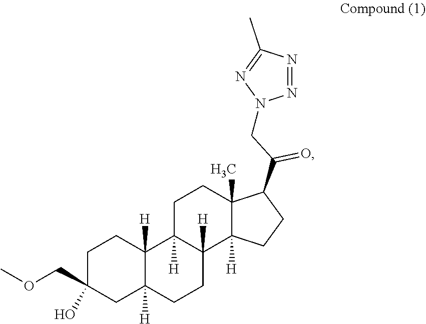

wherein the subject is being treated for a CNS-related disorder.

In one aspect, the present disclosure provides a method of treating a gastrointestinal (GI) disease or condition in a subject in need thereof, comprising administering a therapeutically effective amount of a pharmaceutically acceptable salt of Compound (1):

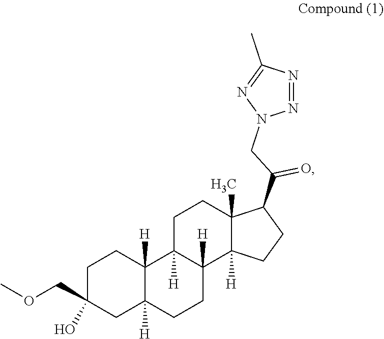

wherein the subject is being treated for a CNS-related disorder.

In one aspect, the present disclosure provides a method of treating irritable bowel syndrome (IBS) in a subject in need thereof, comprising administering a therapeutically effective amount of Compound (1):

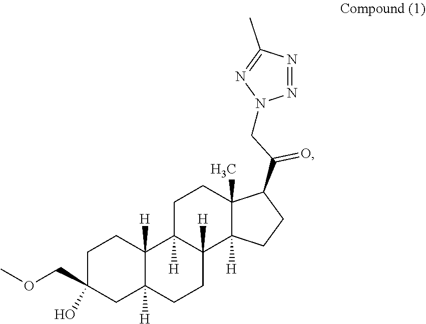

wherein the subject is being treated for a CNS-related disorder.

In one aspect, the present disclosure provides a method of treating irritable bowel syndrome (IBS) in a subject in need thereof, comprising administering a therapeutically effective amount of a pharmaceutically acceptable salt of Compound (1):

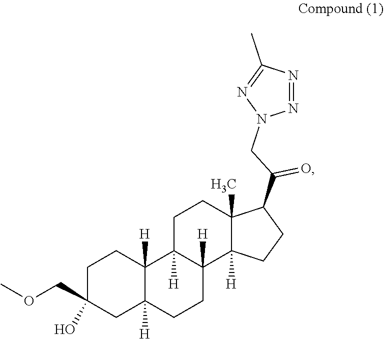

wherein the subject is being treated for a CNS-related disorder.

### Dosage

In some embodiments, Compound (1) is administered at a dose of about 10 mg to about 100 mg. In some embodiments, Compound (1) is administered at a dose of about 10 mg to about 70 mg. In some embodiments, Compound (1) is administered at a dose of about 15 mg to about 75 mg. In some embodiments, Compound (1) is administered at a dose of about 15 mg to about 60 mg. In some embodiments, Compound (1) is administered at a dose of about 20 mg to about 60 mg. In some embodiments, Compound (1) is administered at a dose of about 25 mg to about 55 mg. In some embodiments, Compound (1) is administered at a dose of about 30 mg to about 50 mg. In some embodiments, Compound (1) is administered at a dose of about 40 mg to about 50 mg. In some embodiments, Compound (1) is administered at a dose of about 15 mg, about 20 mg, about 25 mg, about 30 mg, about 35 mg, about 40 mg, about 45 mg, about 50 mg, about 55 mg, or about 60 mg. In some embodiments, Compound (1) is administered at a dose of about 60 mg. In some embodiments, Compound (1) is administered at a dose of about 50 mg.

In some embodiments, Compound (1) is administered at a dose of about 10 mg to about 100 mg per dose per day. In some embodiments, Compound (1) is administered at a dose of about 10 mg to about 70 mg per dose per day. In some embodiments, Compound (1) is administered at a dose of about 15 mg to about 60 mg per dose per day. In some embodiments, Compound (1) is administered at a dose of about 20 mg to about 60 mg per dose per day. In some embodiments, Compound (1) is administered at a dose of about 30 mg to about 50 mg per dose per day. In some embodiments, Compound (1) is administered at a dose of about 25 mg to about 55 mg per dose per day. In some embodiments, Compound (1) is administered at a dose of about 40 mg to about 50 mg per dose per day. In some embodiments, Compound (1) is administered at a dose of about 15 mg, about 20 mg, about 25 mg, about 30 mg, about 35 mg, about 40 mg, about 45 mg, about 50 mg, about 55 mg, or about 60 mg per dose per day. In some embodiments, Compound (1) is administered at a dose of about 60 mg per dose per day. In some embodiments, Compound (1) is administered at a dose of about 50 mg per dose per day.

In some embodiments, Compound (1) is administered once a day for less than 2 weeks. In some embodiments, Compound (1) is administered once a day for about 14 days. In some embodiments, Compound (1) is administered once a day at a dose of about 60 mg once a day for less than 2 weeks. In some embodiments, Compound (1) is administered at a dose of about 60 mg once a day for about 14 days. In some embodiments, Compound (1) is administered at a dose of about 50 mg once a day for less than 2 weeks. In some embodiments, Compound (1) is administered at a dose of about 50 mg once a day for about 14 days.

In some embodiments, the patient is administered Compound (1) at night. In some embodiments, the patient is administered Compound (1) no later than 1 hour before the patient sleeps. In some embodiments, the patient is administered Compound (1) no later than 15 minutes before the patient sleeps.

In some embodiments, Compound (1) is administered with food. In some embodiments, Compound (1) is administered with fat-containing food. Examples of fat-containing food include nuts, peanut butter, avocado, eggs, and cheese. In some embodiments, Compound (1) is administered at night with fat-containing food (e.g., within 1 hour of an evening meal which contains fat, or with a fat-containing snack).

In some embodiments, Compound (1) is administered orally, parenterally, intradermally, intrathecally, intramuscularly, subcutaneously, vaginally, as a buccal, sublingually, rectally, topically, as an inhalation, intranasally, or transdermally. In some embodiments, Compound (1) is administered orally.

In some embodiments, Compound (1) is administered chronically.

In some embodiments, Compound (1) is administered in one or more capsules. In some embodiments, the therapeutically effective amount is administered across two capsules. In some embodiments, the therapeutically effective amount is administered across three capsules.

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 10 mg to about 70 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 15 mg to about 60 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 25 mg to about 55 mg of the free base compound. In some embodiments, the pharmaceutically acceptable salt of Compound (1) is administered at a dose equivalent of about 30 mg to about 50 mg of the free base compound. In some embodiments, the pharmaceutically acceptable salt of Compound (1) is administered at a dose equivalent of about 40 mg to about 50 mg of the free base compound. In some embodiments, the pharmaceutically acceptable salt of Compound (1) is administered at a dose equivalent of about 15 mg, about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 60 mg of the free base compound. In some embodiments, the pharmaceutically acceptable salt of Compound (1) is administered at a dose equivalent of about 50 mg of the free base compound.

In some embodiments, the pharmaceutically acceptable salt of Compound (1) is administered at a dose equivalent of about 10 mg to about 100 mg of the free base compound per dose per day. In some embodiments, the pharmaceutically acceptable salt of Compound (1) is administered at a dose equivalent of about 10 mg to about 70 mg of the free base compound per dose per day. In some embodiments, the pharmaceutically acceptable salt of Compound (1) is administered at a dose equivalent of about 15 mg to about 60 mg of the free base compound per dose per day. In some embodiments, the pharmaceutically acceptable salt of Compound (1) is administered at a dose equivalent of about 20 mg to about 60 mg of the free base compound per dose per day. In some embodiments, the pharmaceutically acceptable salt of Compound (1) is administered at a dose equivalent of about 25 mg to about 55 mg of the free base compound per dose per day. In some embodiments, the pharmaceutically acceptable salt of Compound (1) is administered at a dose equivalent of about 30 mg to about 50 mg of the free base compound per dose per day. In some embodiments, the pharmaceutically acceptable salt of Compound (1) is administered at a dose equivalent of about 40 mg to about 50 mg of the free base compound per dose per day. In some embodiments, the pharmaceutically acceptable salt of Compound (1) is administered at a dose equivalent of about 15 mg, about 20 mg, about 25 mg, about 30 mg, about 35 mg, about 40 mg, about 45 mg, about 50 mg, about 55 mg, or about 60 mg of the free base compound per dose per day. In some embodiments, the pharmaceutically acceptable salt of Compound (1) is administered at a dose equivalent of about 60 mg of the free base compound per dose per day. In some embodiments, the pharmaceutically acceptable salt of Compound (1) is administered at a dose equivalent of about 50 mg of the free base compound per dose per day.

In some embodiments, the pharmaceutically acceptable salt of Compound (1) is administered once a day for less than 2 weeks. In some embodiments, the pharmaceutically acceptable salt of Compound (1) is administered once a day for about 14 days. In some embodiments, the pharmaceutically acceptable salt of Compound (1) is administered at a dose equivalent of about 60 mg of the free base compound once a day for less than 2 weeks. In some embodiments, the pharmaceutically acceptable salt of Compound (1) is administered at a dose equivalent of about 60 mg of the free base compound once a day for about 14 days. In some embodiments, the pharmaceutically acceptable salt of Compound (1) is administered at a dose equivalent of about 50 mg of the free base compound once a day for less than 2 weeks. In some embodiments, the pharmaceutically acceptable salt of Compound (1) is administered at a dose equivalent of about 50 mg of the free base compound once a day for about 14 days.

In some embodiments, the pharmaceutically acceptable salt of Compound (1) is administered at night. In some embodiments, the pharmaceutically acceptable salt of Compound (1) is administered no later than 1 hour before the subject sleeps. In some embodiments, the pharmaceutically acceptable salt of Compound (1) is administered no later than 15 minutes before the subject sleeps. In some embodiments, the pharmaceutically acceptable salt of Compound (1) is administered chronically.

In some embodiments, the pharmaceutically acceptable salt of Compound (1) is administered with food. In some embodiments, the pharmaceutically acceptable salt of Compound (1) is administered with fat-containing food. Examples of fat-containing food include nuts, peanut butter, avocado, eggs, and cheese. In some embodiments, Compound (1) is administered at night with fat-containing food (e.g., within 1 hour of an evening meal which contains fat, or with a fat-containing snack).

In some embodiments, the pharmaceutically acceptable salt of Compound (1) is administered orally, parenterally, intradermally, intrathecally, intramuscularly, subcutaneously, vaginally, as a buccal, sublingually, rectally, topically, as an inhalation, intranasaly, or transdermally. In some embodiments, the pharmaceutically acceptable salt of Compound (1) is administered orally.

In some embodiments, the pharmaceutically acceptable salt of Compound (1) is administered chronically.

### Pharmaceutical Compositions

Another aspect of the disclosure provides a pharmaceutical composition comprising Compound (1) (also referred to as the “active ingredient”), and a pharmaceutically acceptable excipient for use in the combination and methods described herein. In another aspect, the disclosure provides a pharmaceutical composition comprising a pharmaceutically acceptable salt of the active ingredient and a pharmaceutically acceptable excipient for use in the combination and methods described herein. In certain embodiments, the pharmaceutical composition comprises an effective amount of the active ingredient or a pharmaceutically acceptable salt of the active ingredient. In certain embodiments, the pharmaceutical composition comprises a therapeutically effective amount of the active ingredient or a pharmaceutically acceptable salt of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, sublingual/buccal, ocular, otic, vaginal, and intranasal administration or inhalation administration. In some embodiments, the pharmaceutical composition is administered orally.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The pharmaceutical compositions provided herein can also be administered chronically (“chronic administration”). Chronic administration refers to administration of a solid form of Compound (1) or pharmaceutical composition thereof over an extended period of time, e.g., for example, over 3 months, 6 months, 1 year, 2 years, 3 years, 5 years, or the like, or may be continued indefinitely, for example, for the rest of the subject's life. In certain embodiments, the chronic administration is intended to provide a consistent level of Compound (1) in the blood or brain, e.g., within the therapeutic window over the extended period of time.

The pharmaceutical compositions of the present invention may be further delivered using a variety of dosing methods. For example, in certain embodiments, the pharmaceutical composition may be given as an injection, e.g., in order to raise the concentration of Compound (1) in the blood to an effective level. The placement of the injection dose depends on the systemic levels of the active ingredient desired throughout the body, e.g., an intramuscular or subcutaneous injection dose allows a slow release of the active ingredient.

The compositions for oral administration can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions Compound (1) is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg (e.g., from about 0.1 to about 10 mg/kg, from about 0.2 to about 5 mg/kg, from about 0.1 to about 1 mg/kg, from about 0.2 to about 0.8 mg/kg, from about 0.2 to about 0.7 mg/kg, or from about 0.2 to about 0.5 mg/kg) of Compound (1) provided herein. In some instances, Compound (1) is administered at a dosage amount of about 10 mg to about 70 mg (e.g., about 15 mg to about 60 mg, about 25 mg to about 55 mg, or about 30 mg to about 50 mg) per oral dose per day. In other instances, Compound (1) is administered at a dosage amount of about 15 mg, about 25 mg, about 30 mg, about 45 mg, about 50 mg, or about 60 mg per oral dose per day.

Transdermal doses are generally selected to provide similar or lower blood levels than are achieved using injection doses, generally in an amount ranging from about 0.01 to about 20% by weight of, e.g., the drug reservoir or drug-adhesive reservoir for the transdermal patch, preferably from about 0.1 to about 20% by weight, preferably from about 0.1 to about 10% by weight, and more preferably from about 0.5 to about 15% by weight.

Solid compositions may include, for example, any of the following ingredients, or compounds of a similar nature: binders, surfactants, diluents or fillers, buffering agents, anti-adherents, glidants, hydrophilic or hydrophobic polymers, retardants (e.g., delayed release agents), stabilizing agents or stabilizers, disintegrants or superdisintegrants, dispersants, antioxidants, antifoaming agents, fillers, flavors, colorants, lubricants, sorbents, preservatives, plasticizers, coatings, or sweeteners, or mixtures thereof. For example, the excipient or excipients could be a binder such as microcrystalline cellulose, polyvinyl pyrrolidone, hydroxypropyl cellulose, low viscosity hydroxypropyl methylcellulose, gum tragacanth or gelatin; a diluent such as mannitol, microcrystalline cellulose, maltodextrin, starch or lactose, a disintegrating agent such as alginic acid, sodium starch glycolate (e.g., Primogel), croscarmellose sodium, crospovidone, or corn starch; a lubricant such as magnesium stearate, sodium stearyl fumarate or glyceryl behenate; a glidant such as colloidal silicon dioxide or talc; a preservative such as potassium sorbate or methyl paraben, a surfactant, such as sodium lauryl sulfate, docusate sodium, polysorbate 20, polysorbate 80, cetyl triethyl ammonium bromide, polyethyelene oxide-polypropylene oxide copolymers, or Cremophor EL, an antioxidant such as butylhydroxyox toluene, butyl hydroxyanisole, propyl gallate, ascorbic acid, citric acid, tocopherol or tocopherol acetate, sodium sulfite, or sodium metabisulfite, a coating comprising one or more of hydroxypropylmethylcellulose, polyvinyl alcohol, acrylate copolymers, cellulose acetate, ethyl acetate, hydroxypropylmethylcellulose acetate succinate, shellac and others, a sweetening agent such as sucrose, sucralose, acesulfame K, sodium aspartame or saccharin; or a flavoring agent such as peppermint, methyl salicylate, cherry, grape, lemon, or orange flavoring. Any of the well-known pharmaceutical excipients may be incorporated in the dosage form and may be found in the FDA's Inactive Ingredients Guide, Remington: The Science and Practice of Pharmacy, Twenty-first Ed., (Pharmaceutical Press, 2005); Handbook of Pharmaceutical Excipients, Sixth Ed. (Pharmaceutical Press, 2009) all of which are incorporated by reference.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration and stability of the active ingredients or Formulation. All such known transdermal formulations and ingredients are included within the scope provided herein. Topical delivery compositions of interest include liquid formulations, such as lotions (liquids containing insoluble material in the form of a suspension or emulsion, intended for external application, including spray lotions) and aqueous solutions, semi-solid formulations, such as gels (colloids in which the disperse phase has combined with the dispersion medium to produce a semisolid material, such as a jelly), creams (soft solids or thick liquids) and ointments (soft, unctuous preparations), and solid formulations, such as topical patches. As such, delivery vehicle components of interest include, but are not limited to: emulsions of the oil-in-water (O/W) and the water in-oil (W/0) type, milk preparations, lotions, creams, ointments, gels, serum, powders, masks, packs, sprays, aerosols, sticks, and patches.

Compound (1) provided herein can also be administered by a transdermal device. Accordingly, transdermal administration can be accomplished using a patch either of the reservoir or membrane type, or of an adhesive matrix or other matrix variety. Delivery compositions of interest include liquid formulations, such as lotions (liquids containing insoluble material in the form of a suspension or emulsion, intended for external application, including spray lotions) and aqueous solutions, semi-solid formulations, such as gels (colloids in which the disperse phase has combined with the dispersion medium to produce a semisolid material, such as a jelly), creams (soft solids or thick liquids) and ointments (soft, unctuous preparations), and solid formulations, such as topical patches. As such, delivery vehicle components of interest include, but are not limited to: emulsions of the oil-in-water (O/W) and the water in-oil (W/O) type, milk preparations, lotions, creams, ointments, gels, serum, powders, masks, packs, sprays, aerosols, sticks, and patches. For a transdermal patch, the active agent layer includes one or more active agents, one of which is Compound (1). In certain embodiments, the matrix is an adhesive matrix. The matrix may include polymeric materials. Suitable polymers for the adhesive matrix include, but are not limited to: polyurethanes, acrylates, styrenic block copolymers, silicones, and the like. For example, the adhesive matrix may include, but is not limited to, an acrylate polymer, polysiloxanes, polyisobutylene (PIB), polyisoprene, polybutadiene, styrenic block polymers, combinations of thereof, and the like. Additional examples of adhesives are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference.

In certain embodiments, the active agent layer includes a permeation enhancer. The permeation enhancer may include, but is not limited to the following: aliphatic alcohols, such as but not limited to saturated or unsaturated higher alcohols having 12 to 22 carbon atoms, such as oleyl alcohol and lauryl alcohol; fatty acids, such as but not limited to linolic acid, oleic acid, linolenic acid, stearic acid, isostearic acid and palmitic acid; fatty acid esters, such as but not limited to isopropyl myristate, diisopropyl adipate, and isopropyl palmitate; alcohol amines, such as but not limited to triethanolamine, triethanolamine hydrochloride, and diisopropanolamine; polyhydric alcohol alkyl ethers, such as but not limited to alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as but not limited to polyoxyethylene alkyl ethers in which the number of carbon atoms of the alkyl group moiety is 6 to 20, and the number of repeating units (e.g., —OCH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (e.g., fatty acid esters of glycerol), such as but not limited to glycerol esters of fatty acids having 6 to 18 carbon atoms, diglycerides, triglycerides or combinations thereof. In some embodiments, the polymer matrix includes a polyvinylpyrrolidone. The composition may further include one or more fillers or one or more antioxidants. In some embodiments, the transdermal formulations described may have a multi-layer structure. For example, the transdermal formulation may have an adhesive matrix and a backing.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

Compound (1) can also be administered in sustained release forms or from sustained release drug delivery systems. A description of representative sustained release materials can be found in Remington's Pharmaceutical Sciences

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21st ed., Lippincott Williams & Wilkins, 2005.

### Kits

Another aspect of the disclosure provides a kit comprising a pharmaceutical composition comprising Compound (1) and an instruction set describing a method for treating a gastrointestinal (GI) disease or condition in a subject.

Another aspect of the disclosure includes a kit comprising a plurality of therapeutically efficacious dosages of Compound (1) and an instruction set describing a method of administering the dosages for treating a gastrointestinal (GI) disease or condition in a subject. In some embodiments, the method of administering the dosages is any one of the methods described herein.

In some embodiments, the dosages of Compound (1) are individual dosage units of Compound (1). In some embodiments, an individual dosage unit comprises from about 10 mg to about 70 mg (e.g., about 15 mg to about 60 mg, about 25 mg to about 55 mg, or about 30 mg to about 50 mg) of Compound (1). In some embodiments, the individual dosage unit of Compound (1) is a capsule or a tablet. In some embodiments, the plurality of therapeutically efficacious dosages of Compound (1) comprises two capsules or tablets. In some embodiments, the plurality of therapeutically efficacious dosages of Compound (1) comprises three capsules or tablets.

In some embodiments, the instruction set is printed on a suitable material. In some embodiments, the instruction set indicates that the method further comprises administration of a plurality of therapeutically efficacious dosages of an additional therapeutic agent. In some embodiments, the dosages of the additional therapeutic agent are individual dosage units of the additional therapeutic agent. The additional therapeutic agent is any one of the agents described herein and combinations thereof.

### Other Embodiments

Embodiments of the present subject matter disclosed herein may be beneficial alone or in combination with one or more other embodiments. Without limiting the foregoing description, certain non-limiting embodiments of the disclosure, numbered 1-46 are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### EMBODIMENTS OF THE DISCLOSURE

Embodiment 1. A method of treating a gastrointestinal (GI) disease or condition in a subject in need thereof, comprising administering a therapeutically effective amount of Compound (1):

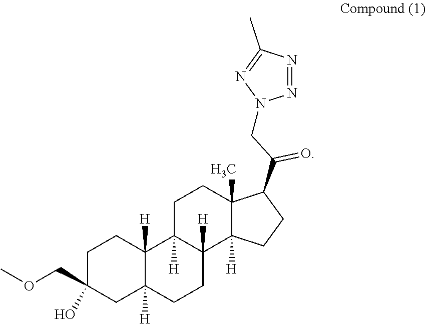

Embodiment 2. A method of treating a gastrointestinal (GI) disease or condition in a subject in need thereof, comprising administering a therapeutically effective amount of a pharmaceutically acceptable salt of Compound (1):

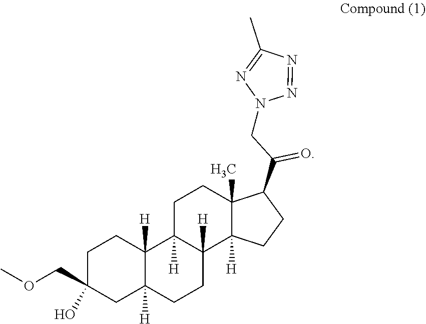

Embodiment 3. The method of Embodiment 1 or Embodiment 2, wherein the GI disease or condition is a non-inflammatory GI disease or condition.

Embodiment 4. The method of Embodiment 1 or Embodiment 2, wherein the GI disease or condition is an inflammatory GI disease or condition.

Embodiment 5. The method of any one of Embodiments 1-4, wherein the GI disease or condition is inflammatory bowel disease (IBD), ulcerative colitis (UC), Crohn's disease (CD), irritable bowel syndrome (IBS), indeterminate colitis (IC), microscopic colitis, collagenous colitis, lymphocytic colitis, incomplete microscopic colitis, segmental colitis associated with diverticula (SCAD), or combinations thereof.

Embodiment 6. The method of Embodiment 5, wherein the GI disease or condition is irritable bowel syndrome (IBS).

Embodiment 7. The method of Embodiment 6, wherein the IBS is IBS with constipation (IBS-C), IBS with diarrhea (IBS-D), IBS with mixed constipation and diarrhea (IBS-M), IBS with alternating stool pattern (IBS-A), post-infection (PI) IBS, or post diverticulitis IBS.

Embodiment 8. The method of any one of Embodiments 1-7, wherein the subject has a CNS-related disorder.

Embodiment 9. The method of any one of Embodiments 1-7, wherein the subject is being treated for a CNS-related disorder.

Embodiment 10. The method of Embodiment 8 or 9, wherein the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus.

Embodiment 11. The method of Embodiment 10, wherein the CNS-related disorder is a mood disorder.

Embodiment 12. The method of Embodiment 11, wherein the mood disorder is major depressive disorder or postpartum depression.

Embodiment 13. The method of Embodiment 9, wherein the subject is being treated with an antidepressant.

Embodiment 14. The method of Embodiment 13, wherein the antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 15. The method of Embodiment 13, wherein the antidepressant is 4-Chlorokynurenine (AV-101), Apimostinel (NRX-1074), Arketamine (PCN-101, HR-071603), Dextromethadone (REL-1017), MU-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), PRAX-114, Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 3β-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05), Carbidopa/oxitriptan (EVX-101), Cycloserine/lurasidone (NRX-101; Cyclurad), or Deudextromethorphan/quinidine (AVP-786, CTP-786).

Embodiment 16. The method of any one of Embodiments 1-15, wherein the method results in the attenuation of at least one clinical feature associated with a GI disease or condition.

Embodiment 17. The method of Embodiment 16, wherein the at least one clinical feature associated with a GI disease or condition is inflammation, jaundice, rectal bleeding, urgency, diarrhea, tenesmus, incontinence, fistula formation, constipation, bloating, abdominal cramps, colicky abdominal pain, change in bowel habit, mouth ulcers, anemia with associated symptoms of palpitations, dizziness, and dyspnea, fistulae, nausea, vomiting, fatigue, malaise, fever, or loss of weight or appetite.

Embodiment 18. The method of any one of Embodiments 4-17, wherein the method reduces the severity and/or duration of at least one symptom associated with the inflammatory disease or condition.

Embodiment 19. The method of any one of Embodiments 1-18, wherein the method further comprises administration of an additional therapeutic agent.

Embodiment 20. The method of Embodiment 19, wherein the additional therapeutic agent is an anti-inflammatory agent, an immunosuppressant drug, an 5-aminosalicylate (5-ASA), a corticosteroid, a tumor necrosis factor (TNF)-alpha inhibitor, an alpha-4 integrin inhibitor, an IL-12 and IL-23 inhibitor, a biologic, a biosimilar, an antibiotic, a dietary supplement, a laxative, an antispasmodic, an antidepressant, anti-diarrheal medication, a pain medication, or combinations thereof.

Embodiment 21. The method of Embodiment 20, wherein the additional therapeutic is adalimumab (Humira®), adalimumab-atto (Amjevita®), alosetron (Lotronex®), ampicillin (Omnipen®), azathioprine (Azasan®, Imuran®), balsalazide (Colazal®, Giazol®), cyclosporine (Gengraf®, Neoral®, Sandimmune®), certolizumab (Cimzia®), ciprofloxacin (Cipro®), eluxadoline (Viberzi®), golimumab (Simponi®), infliximab (Remicade®), infliximab-dyyb (Inflectra®), linaclotide (Linzess®), lubiprostone (Amitiza®), mesalamine (Asacol®, Canasa®, Delzicol®, Lialda®, Pentasa®), 6-mercaptopurine (Purinethol®, Purixan®), methotrexate (Trexall®, MTX®, Rheumatrex®, Mexate®), methylprednisolone, metronidazole (Flagyl®), natalizumab (Tysabri®), olsalazine (Dipentum®), prednisone, rifaximin (Xifaxan®), sulfasalazine (Azulfidine®), tacrolimus (Prograf®), tetracycline, ustekinumab (Stelara®), vedolizumab (Entyvio®), or combinations thereof.

Embodiment 22. The method of any one of Embodiments 1-21, wherein Compound (1), or a pharmaceutically acceptable salt thereof, is administered once a day.

Embodiment 23. The method of Embodiment 1, wherein Compound (1) is administered at a dose of about 20 mg, about 25 mg, about 30 mg, about 35 mg, about 40 mg, about 45 mg, about 50 mg, about 55 mg, or about 60 mg.

Embodiment 24. The method of Embodiment 23, wherein Compound (1) is administered at a dose of about 50 mg.

Embodiment 25. The method of Embodiment 23, wherein Compound (1) is administered at a dose of about 60 mg.

Embodiment 26. The method of Embodiment 24, wherein Compound (1) is administered at a dose of about 50 mg once a day.

Embodiment 27. The method of Embodiment 25, wherein Compound (1) is administered at a dose of about 60 mg once a day.

Embodiment 28. The method of Embodiment 2, wherein the pharmaceutically acceptable salt of Compound (1) is administered at a dose equivalent to about 20 mg, about 25 mg, about 30 mg, about 35 mg, about 40 mg, about 45 mg, about 50 mg, about 55 mg, or about 60 mg of the free base compound.

Embodiment 29. The method of Embodiment 28, wherein the pharmaceutically acceptable salt of Compound (1) is administered at a dose equivalent to about 50 mg of the free base compound.

Embodiment 30. The method of Embodiment 28, wherein the pharmaceutically acceptable salt of Compound (1) is administered at a dose equivalent to about 60 mg of the free base compound.

Embodiment 31. The method of Embodiment 29, wherein the pharmaceutically acceptable salt of Compound (1) is administered at a dose equivalent to about 50 mg of the free base compound once a day.

Embodiment 32. The method of Embodiment 30, wherein the pharmaceutically acceptable salt of Compound (1) is administered at a dose equivalent to about 60 mg of the free base compound once a day.

Embodiment 33. The method of any one of Embodiments 1-32, wherein Compound
(1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasally, or transdermally.

Embodiment 34. The method of Embodiment 33, wherein Compound (1), or the pharmaceutically acceptable salt of Compound (1), is administered orally.

Embodiment 35. The method of any one of Embodiments 1-34, wherein Compound (1), or the pharmaceutically acceptable salt of Compound (1), is administered with food.

Embodiment 36. The method of any one of Embodiments 1-35, wherein Compound (1), or the pharmaceutically acceptable salt of Compound (1), is administered once a day at night.

Embodiment 37. A method of treating a gastrointestinal (GI) disease or condition in a subject in need thereof, comprising administering about 20 mg to about 60 mg of Compound (1):

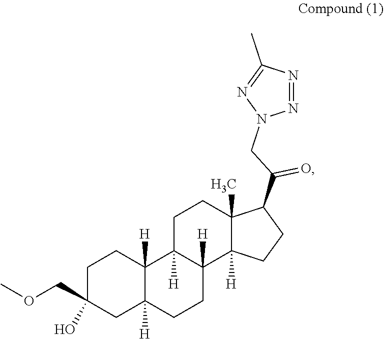

Embodiment 38. A method of treating a gastrointestinal (GI) disease or condition in a subject in need thereof, comprising administering a pharmaceutically acceptable salt of Compound (1):

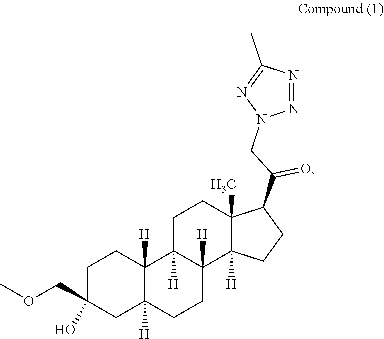

at a dose equivalent of from about 20 mg to about 60 mg of the free base compound.

Embodiment 39. A method of treating irritable bowel syndrome (IBS) in a subject in need thereof, comprising administering a therapeutically effective amount of Compound (1):

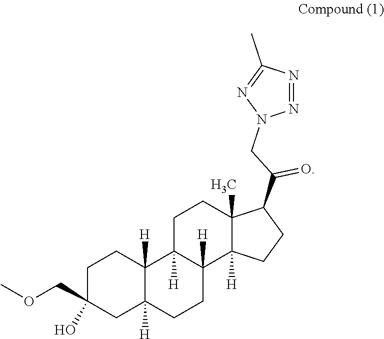

Embodiment 40. A method of treating irritable bowel syndrome (IBS) in a subject in need thereof, comprising administering a therapeutically effective amount of a pharmaceutically acceptable salt of Compound (1):

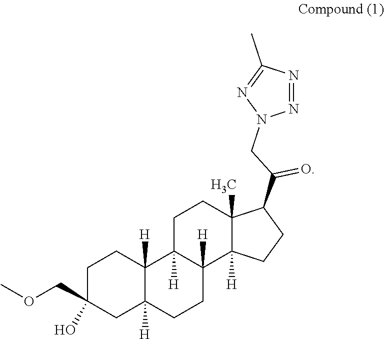

Embodiment 41. A method of treating irritable bowel syndrome (IBS) in a subject in need thereof, comprising administering about 20 mg to about 60 mg of Compound (1):

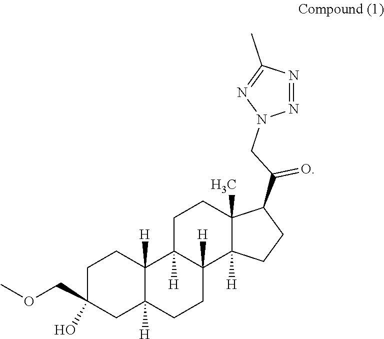

Embodiment 42. A method of treating irritable bowel syndrome (IBS) in a subject in need thereof, comprising administering a pharmaceutically acceptable salt of Compound (1) at a dose equivalent to about 20 mg to about 60 mg of the free base compound:

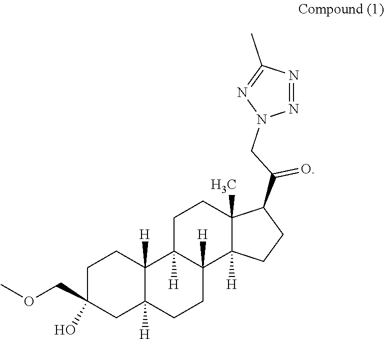

Embodiment 43. A method of treating a gastrointestinal (GI) disease or condition in a subject in need thereof, comprising administering a therapeutically effective amount of Compound (1):

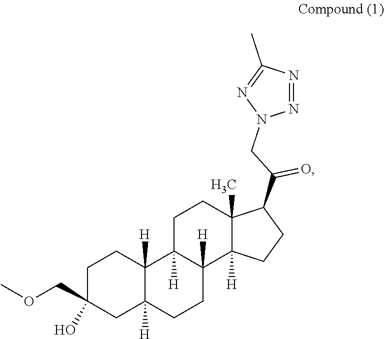

wherein the subject is being treated for a CNS-related disorder.

Embodiment 44. A method of treating a gastrointestinal (GI) disease or condition in a subject in need thereof, comprising administering a therapeutically effective amount of a pharmaceutically acceptable salt of Compound (1):

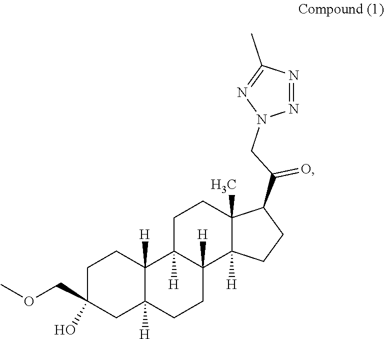

wherein the subject is being treated for a CNS-related disorder.

Embodiment 45. A method of treating irritable bowel syndrome (IBS) in a subject in need thereof, comprising administering a therapeutically effective amount of Compound (1):

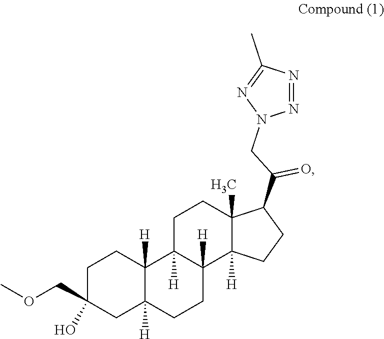

wherein the subject is being treated for a CNS-related disorder.

Embodiment 46. A method of treating irritable bowel syndrome (IBS) in a subject in need thereof, comprising administering a therapeutically effective amount of a pharmaceutically acceptable salt of Compound (1):

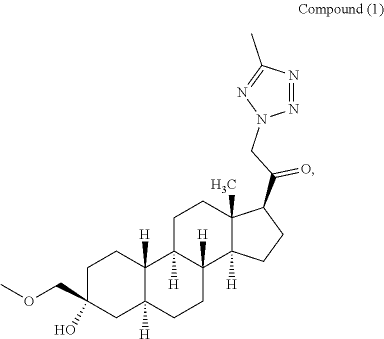

wherein the subject is being treated for a CNS-related disorder.

### EXAMPLES

In order that the invention described herein may be more fully understood, the following examples are set forth. The examples described in this application are offered to illustrate the methods provided herein and are not to be construed in any way as limiting their scope.

Compound (1) and zuranolone, which is the compound that is described in the Examples below, are neuroactive steroids that have been shown to be positive allosteric modulators of γ-aminobutyric acid type A (GABA_{A}) receptors that target synaptic and extrasynaptic GABA_{A} receptors. As positive allosteric modulators of GABA_{A} receptors, Compound (1) and zuranolone may serve as therapeutic agents to treat CNS-related disorders, e.g., depression, postpartum depression, and major depressive disorder.

### Example 1: Treatment of MDD with Zuranolone Co-Initiated with an Antidepressant

A randomized, double-blind, parallel-group, active/placebo-controlled study (NCT04476030) in participants with major depressive disorder (MDD) was conducted. The study evaluated the efficacy and safety of zuranolone 50 mg co-initiated with standard-of-care (SOC) antidepressant therapies (ADTs) (zuranolone+ADT) vs placebo co-initiated with SOC ADT (placebo+ADT) in adults with MDD. Patients were randomized 1:1 to once-daily, blinded zuranolone+ADT or placebo+ADT for 14 days then continued open-label SOC ADT for 28 more days.

The study met its objectives, demonstrating a rapid and statistically significant reduction in depressive symptoms at Day 3 and over the 2-week treatment period, achieving the primary and key secondary endpoints. The primary endpoint was change from baseline (CFB) in the 17-item Hamilton Rating Scale for Depression (HAMD-17) total score at Day 3. Among 425 patients in the full analysis set, CFB in HAMD-17 total score at Day 3 was significantly greater with zuranolone+ADT vs placebo+ADT (least squares [LS] mean [standard error (SE)], −8.9 [0.39] vs −7.0 [0.38]; p=0.0004).

Zuranolone+ADT was generally well-tolerated, with no new safety signals identified.

### Adverse and Serious Adverse Events

### Adverse Event (AE)

An AE was any untoward medical occurrence in a patient or clinical investigation participant administered a pharmaceutical product and that did not necessarily have a causal relationship with this treatment. An AE could therefore be any unfavorable and unintended sign (including an abnormal laboratory finding), symptom or disease temporally associated with the use of a medicinal (investigational) product whether or not related to the medicinal (investigational) product. In clinical studies, an AE could include an undesirable medical condition occurring at any time, including baseline or washout periods, even if no study treatment had been administered.

A treatment emergent adverse event (TEAE) was defined as an AE with onset after the start of investigational product (IP), or any worsening of a pre-existing medical condition/AE with onset after the start of IP and throughout the study. The term IP included any Sage IP, a comparator, or a placebo administered in a clinical study.

Laboratory abnormalities and changes from baseline in vital signs, and electrocardiograms (ECGs) were considered AEs if they resulted in discontinuation or interruption of study treatment, required therapeutic medical intervention, met protocol specific criteria (if applicable) or if the investigator considered them to be clinically significant. Any abnormalities that met the criteria for a serious adverse event (SAE) should have been reported in an expedited manner. Laboratory abnormalities and changes from baseline in vital signs and ECGs that were clearly attributable to another AE did not require discrete reporting (e.g., electrolyte disturbances in the context of dehydration, chemistry and hematologic disturbances in the context of sepsis).

All AEs that occurred after any participant had signed the informed consent and throughout the duration of the study, whether or not they were related to the study, must have been reported to Sage Therapeutics.

Participants who discontinued the IP due to an AE, regardless of investigator-determined causality, should have been followed until the event was resolved, considered stable, or the investigator determined the event was no longer clinically significant. Any AEs that were unresolved at the participant's last AE assessment in the study were followed up by the investigator for as long as medically indicated, but without further recording in the electronic case report form (eCRF).

### Serious Adverse Event (SAE)

An SAE was any untoward medical occurrence that at any dose:
Resulted in death;
Placed the participant at immediate risk of death (a life-threatening event); however, this did not include an event that, had it occurred in a more severe form, might have caused death;
Required inpatient hospitalization or prolongation of existing hospitalization;
Resulted in persistent or significant disability or incapacity; or
Resulted in a congenital abnormality or birth defect.

An SAE may also been any other medically important event that, in the opinion of the investigator may jeopardize the participant or may require medical intervention to prevent 1 of the outcomes listed above (examples of such events include allergic bronchospasm requiring intensive treatment in an emergency room or convulsions occurring at home that do not require an inpatient hospitalization).

All SAEs that occurred after any participant has signed the informed consent form (ICF) and throughout the duration of the study, whether or not they were related to the study, must have been recorded on the SAE report form provided by Sage Therapeutics. Any SAE that was ongoing when the participant completed their final study visit, was followed by the investigator until the event had resolved, stabilized, returned to baseline status, or until the participant died or was lost to follow up.

A prescheduled or elective procedure or routinely scheduled treatment was not be considered an SAE, even if the participant was hospitalized.

### Relationship to Investigational Product

The investigator made the determination of relationship to the IP for each AE (not related, related). The following definitions should have been considered when evaluating the relationship of AEs and SAEs to the IP.

Not Related—An AE was considered “not related” to the use of the IP if there was not a reasonable possibility that the event had been caused by the IP. Factors that pointed towards this assessment included but were not limited to: the lack of temporal relationship between administration of the IP and the event, the presence of biologically implausible relationship between the product and the AE, or the presence of a more likely alternative explanation for the AE.

Related—An AE was considered “related” to the use of the IP if there was a reasonable possibility that the event may have been caused by the product under investigation. Factors that pointed towards this assessment included but are not limited to: a positive rechallenge, a reasonable temporal sequence between administration of the drug and the event, a known response pattern of the suspected drug, improvement following discontinuation or dose reduction, a biologically plausible relationship between the drug and the AE, or a lack of alternative explanation for the AE.

### Results

Zuranolone+ADT was generally well-tolerated with no new safety concern or trend identified.

At least one TEAE was reported in 74.1% of patients who received zuranolone+ADT and in 65.6% of patients who received placebo+ADT (Table 1). Most TEAEs experienced by patients were mild or moderate. No deaths occurred. The most common TEAEs (present in ≥10% of patients in zuranolone+ADT vs placebo+ADT groups) were somnolence (18.4% vs 8.3%), dizziness (13.2% vs 7.3%), headache (14.7% vs 11.8%), and nausea (9.0% vs 23.4%). Two serious TEAEs were reported in this study, both in the zuranolone+ADT group (seizure-like phenomenon at Day 7 that was considered related to blinded zuranolone by the investigator and exacerbation of chronic obstructive pulmonary disease at Day 23 that was considered not related to blinded zuranolone or the ADT [sertraline]).

### Severe Treatment-Emergent Adverse Events

Two (0.9%) patients in the zuranolone+ADT group experienced a serious treatment emergent adverse event. Both participants were assigned to a SSRI. One patient experienced seizure-like phenomena approximately 1 hour after receipt of zuranolone 50 mg on Day 7; the event was assessed as related to zuranolone by the investigator with no contributing factors for the event. Zuranolone was discontinued and the event resolved within 22 minutes. The patient completed escitalopram 10 mg dosing and the study.

The other patient experienced exacerbation of chronic obstructive pulmonary disease on Day 23 of sertraline 100 mg dosing during the ADT Continuation Period. The event resolved on Day 25 and was assessed as not related to ADT by the investigator.

No increases from baseline in suicidal ideation/behavior signals, as assessed by the C-SSRS, or withdrawal symptoms, as assessed by the PWC-20, were identified in patients who received zuranolone+ADT. The proportion of patients who had suicidal ideation at any post baseline visit from Day 3 through Day 42 ranged from 4.8% to 11.7% in the zuranolone+ADT group and from 6.0% to 12.4% in the placebo+ADT group. No patients who received placebo+ADT experienced suicidal behavior. For patients who received zuranolone+ADT, one experienced suicidal behavior at baseline and a different patient experienced suicidal behavior at Day 28. Mean decreases from baseline in PWC-20 total score were observed at Days 18 (−0.6 for zuranolone+ADT patients, −1.1 for placebo+ADT patients), Day 21 (−0.8, −1.0) and were similar between the treatment groups.

The incidence of treatment emergent adverse events (TEAEs) in the zuranolone+ADT group was 74.1% (157/212) vs 65.6% (143/218) in the placebo+ADT group. During the double-blind treatment period: 68.4% (145/212) in the zuranolone+ADT group vs 59.2% (129/218) in the placebo+ADT. During the ADT continuation period: 31.6% (67/212) in the zuranolone+ADT group vs 27.5% (60/218) in the placebo+ADT. The majority of participants in both treatment groups experienced TEAEs that were mild to moderate in intensity.

TABLE 1
Summary of Treatment-Emergent Adverse Events by
Maximum Severity and other Safety Parameters.^{a}

| | Placebo + ADT | | Zuranolone + ADT | |
|---|---|---|---|---|
| Safety Parameters | (n = 218) | | (n = 212) | |
| Any TEAE^{b} | 143 | (65.6%) | 157 | (74.1%) |
| Mild | 83 | (38.1%) | 76 | (35.8%) |
| Moderate | 55 | (25.2%) | 73 | (34.4%) |
| Severe | 5 | (2.3%) | 8 | (3.8%) |
| Dose reduction due to TEAE | 6 | (2.8%) | 20 | (9.4%) |
| Treatment discontinuation due to | 8 | (3.7%) | 14 | (6.6%) |
| TEAE | | | | |
| Study withdrawal due to TEAE | 10 | (4.6%) | 11 | (5.2%) |

| | | | |
|---|---|---|---|
| Serious AEs | 0 | 2 | (0.9%)^{c} |

^{a}TEAEs were reported for the safety set. The safety set was defined as all randomized patients administered blinded zuranolone 50 mg or placebo. TEAEs were ordered by descending incidence in the Zuranolone + ADT group.
^{b}Maximum severity of the TEAE.
^{c}One patient experienced a seizure-like phenomenon approximately 1 hour after receipt of zuranolone 50 mg on Day 7; the event was assessed as related to zuranolone. Another patient experienced exacerbation of their chronic obstructive pulmonary disease on Day 23 of the ADT continuation period; the event was assessed as not related to zuranolone or ADT and resolved two days later.

The majority of the TEAEs leading to study drug discontinuation and withdrawal from study in the zuranolone+ADT group were due to TEAEs within the Nervous systems disorders, consistent with the zuranolone data previously reported.

The most common TEAEs in the zuranolone+ADT group (observed in more than 5% participants in either treatment group) are presented below in Table 2.

TABLE 2
Summary of the Most Common (>5% in Any Treatment
Group) Treatment-Emergent Adverse Events (Safety Set).

| | | |
|---|---|---|
| | Placebo + ADT | Zuranolone + ADT |
| Preferred Term | (n = 218) | (n = 212) |

| | | | | |
|---|---|---|---|---|
| Any TEAE | 143 | (65.6%) | 157 | (74.1%) |
| Somnolence | 18 | (8.3%) | 39 | (18.4%) |
| Dizziness | 16 | (7.3%) | 28 | (13.2%) |
| Headache | 32 | (14.7%) | 25 | (11.8%) |
| Insomnia | 17 | (7.8%) | 21 | (9.9%) |
| Dry Mouth | 19 | (8.7%) | 20 | (9.4%) |
| Nausea | 51 | (23.4%) | 19 | (9.0%) |
| Fatigue | 11 | (5.0%) | 18 | (8.5%) |
| Diarrhea | 21 | (9.6%) | 13 | (6.1%) |
| Decreased Appetite | 7 | (3.2%) | 12 | (5.7%) |
| Sedation | 6 | (2.8%) | 12 | (5.7%) |
| Tremor | 3 | (1.4%) | 11 | (5.2%) |

Over the study period, TEAEs ≥10% in either treatment group (zuranolone 50 mg co-initiated with an ADT vs placebo co-initiated with an ADT) were somnolence, dizziness, headache, and nausea as shown in the Table 3 below.

TABLE 3
TEAEs Incidence with ADT and ADT with Zuranolone.

| | |
|---|---|
| Placebo + ADT | Zuranolone + ADT |
| (N = 218)¹ n(%) | (N = 212)¹ n(%) |

| | | | | |
|---|---|---|---|---|
| Somnolence | 18 | (8.3) | 39 | (18.4) |
| Dizziness | 16 | (7.3) | 28 | (13.2) |
| Headache | 32 | (14.7) | 25 | (11.8) |
| Nausea | 51 | (23.4) | 19 | (9.0) |

¹N value is based on the safety set.

No signals for an evidence of withdrawal symptoms were observed after discontinuation of zuranolone+ADT, as assessed by the Physician Withdrawal Checklist (PWC-20). No signals for an increased suicidal ideation or suicidal behavior were observed compared to baseline (as measured by C-SSRS), within the zuranolone+ADT group compared to placebo+ADT group.

Treatment with zuranolone co-initiated with an SOC ADT was generally well tolerated, with safety signals consistent with those reported in previous studies of zuranolone. While patients in both treatment groups experienced mild to moderate AEs consistent with the known safety profiles of the study drugs, a lower incidence of gastrointestinal AEs (e.g., nausea, diarrhea) was observed with zuranolone+ADT vs placebo+ADT.

TEAEs Commonly Associated with SSRI/SNRI

Certain TEAEs that are known to be associated with SSRIs and SNRIs, are presented below. Given the study design (zuranolone co-initiated with an ADT), these TEAE categories were reviewed as those of interest in relevance to ADTs.

Sleep Disorders: 12.7% (27/212) in zuranolone+ADT group versus 9.6% (21/218) in placebo+ADT group
Double blinded treatment period:
   9.9% (21/212) in the zuranolone+ADT group
   9.2% (20/218) in the placebo+ADT group
ADT continuation period:
   3.8% (8/212) in the zuranolone+ADT group
   0.5% (1/218) in the placebo+ADT group

Sexual Dysfunction: 4.7% (10/212) in zuranolone+ADT group versus 3.7% (8/218) in placebo+ADT group
Double blinded treatment period:
   3.8% (8/212) in the zuranolone+ADT group
   3.2% (7/218) in the placebo+ADT group
ADT continuation period:
   0.9% (2/212) in the zuranolone+ADT group
   0.5% (1/218) in the placebo+ADT group

Gastrointestinal disorder: 29.7% (63/212) in zuranolone+ADT group versus 43.1% (94/218) in placebo+ADT group.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Although the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A method of treating a gastrointestinal (GI) disease or condition in a subject in need thereof, comprising administering a therapeutically effective amount of Compound (1), or a pharmaceutically acceptable salt thereof,

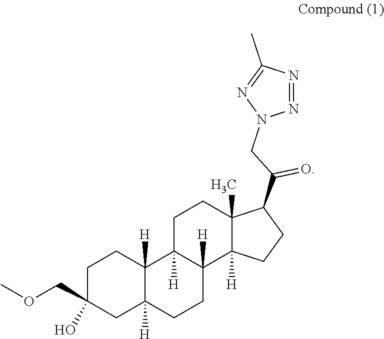

2. The method of claim 1, wherein
a. the GI disease or condition is a non-inflammatory GI disease or condition; or
b. the GI disease or condition is an inflammatory GI disease or condition.

3. The method of claim 1 or 2, wherein the GI disease or condition is inflammatory bowel disease (IBD), ulcerative colitis (UC), Crohn's disease (CD), irritable bowel syndrome (IBS), indeterminate colitis (IC), microscopic colitis, collagenous colitis, lymphocytic colitis, incomplete microscopic colitis, segmental colitis associated with diverticula (SCAD), or combinations thereof,
optionally, wherein the GI disease or condition is irritable bowel syndrome (IBS); and
optionally, wherein the IBS is IBS with constipation (IBS-C), IBS with diarrhea (IBS-D), IBS with mixed constipation and diarrhea (IBS-M), IBS with alternating stool pattern (IBS-A), post-infection (PI) IBS, or post diverticulitis IBS.

4. The method of any one of claims 1-3, wherein the subject has a CNS-related disorder and/or the subject is being treated for a CNS-related disorder;
optionally, wherein the CNS-related disorder is selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, tinnitus, and status epilepticus;
optionally, wherein the CNS-related disorder is a mood disorder; and
optionally, wherein the mood disorder is major depressive disorder or postpartum depression.

5. The method of claim 4, wherein the subject is being treated with an antidepressant;
optionally, wherein the antidepressant is
a. a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine; or
b. 4-Chlorokynurenine (AV-101), Apimostinel (NRX-1074), Arketamine (PCN-101, HR-071603), Dextromethadone (REL-1017), MU-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), PRAX-114, Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 30-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05; Auvelity™), Carbidopa/oxitriptan (EVX-101), Cycloserine/lurasidone (NRX-101; Cyclurad), or Deudextromethorphan/quinidine (AVP-786, CTP-786).

6. The method of any one of claims 1-5, wherein the method results in the attenuation of at least one clinical feature associated with a GI disease or condition;
optionally, wherein the at least one clinical feature associated with a GI disease or condition is inflammation, jaundice, rectal bleeding, urgency, diarrhea, tenesmus, incontinence, fistula formation, constipation, bloating, abdominal cramps, colicky abdominal pain, change in bowel habit, mouth ulcers, anemia with associated symptoms of palpitations, dizziness, and dyspnea, fistulae, nausea, vomiting, fatigue, malaise, fever, or loss of weight or appetite.

7. The method of any one of claims 2-6, wherein the method reduces the severity and/or duration of at least one symptom associated with the inflammatory disease or condition.

8. The method of any one of claims 1-7, wherein the method further comprises administration of an additional therapeutic agent;
optionally, wherein the additional therapeutic agent is:
a. an anti inflammatory agent, an immunosuppressant drug, an 5-aminosalicylate (5-ASA), a corticosteroid, a tumor necrosis factor (TNF)-alpha inhibitor, an alpha-4 integrin inhibitor, an IL-12 and IL-23 inhibitor, a biologic, a biosimilar, an antibiotic, a dietary supplement, a laxative, an antispasmodic, an antidepressant, anti-diarrheal medication, a pain medication, or combinations thereof, or
b. adalimumab (Humira®), adalimumab-atto (Amjevita®), alosetron (Lotronex®), ampicillin (Omnipen®), azathioprine (Azasan®, Imuran®), balsalazide (Colazal®, Giazol®), cyclosporine (GengrafM, Neoral®, Sandimmune®), certolizumab (Cimzia®), ciprofloxacin (Cipro®), eluxadoline (Viberzi®), golimumab (Simponi®), infliximab (Remicade®), infliximab-dyyb (Inflectra®), linaclotide (Linzess®), lubiprostone (Amitiza®), mesalamine (Asacol®, Canasa®, Delzicol®, Lialda®, Pentasa®), 6-mercaptopurine (Purinethol®, Purixan®), methotrexate (Trexall®, MTX®, Rheumatrex®, Mexate®), methylprednisolone, metronidazole (Flagyl®), natalizumab (Tysabri®), olsalazine (Dipentum®), prednisone, rifaximin (Xifaxan®), sulfasalazine (Azulfidine®), tacrolimus (Prograf®), tetracycline, ustekinumab (Stelara®), vedolizumab (Entyvio®), or combinations thereof.

9. The method of any one of claims 1-8, wherein Compound (1), or a pharmaceutically acceptable salt thereof, is administered once a day.

10. The method of any one of claims 1-9, wherein Compound (1) is administered.

11. The method of claim 10, wherein Compound (1) is administered
a. at a dose of about 20 mg, about 25 mg, about 30 mg, about 35 mg, about 40 mg, about 45 mg, about 50 mg, about 55 mg, or about 60 mg;
b. at a dose of about 50 mg;
c. at a dose of about 60 mg;
d at a dose of about 50 mg once a day; or
e. at a dose of about 60 mg once a day.

12. The method of any one of claims 1-9, wherein a pharmaceutically acceptable salt of Compound (1) is administered.

13. The method of claim 13, wherein the pharmaceutically acceptable salt of Compound (1) is administered
a. at a dose equivalent to about 20 mg, about 25 mg, about 30 mg, about 35 mg, about 40 mg, about 45 mg, about 50, about 55 mg, or about 60 mg of the free base compound;
b. at a dose equivalent to about 50 mg of the free base compound;
c. at a dose equivalent to about 60 mg of the free base compound;
d. at a dose equivalent to about 50 mg of the free base compound once a day; or
e. at a dose equivalent to about 60 mg of the free base compound once a day.

14. The method of any one of claims 1-13, wherein Compound (1), or the pharmaceutically acceptable salt thereof, is administered orally, parenterally, intradermally, intrathecally, intramuscularly, subcutaneously, vaginally, as a buccal, sublingually, rectally, topically, as an inhalation, intranasally, or transdermally;
optionally, wherein Compound (1), or the pharmaceutically acceptable salt thereof, is administered orally;
optionally, wherein Compound (1), or the pharmaceutically acceptable salt thereof, is administered with food; and/or
optionally, wherein Compound (1), or the pharmaceutically acceptable salt thereof, is administered once a day at night.

15. A method of treating a gastrointestinal (GI) disease or condition in a subject in need thereof, comprising administering from about 20 mg to about 60 mg of Compound (1), or a pharmaceutically acceptable salt of Compound (1) at a dose equivalent of from about 20 mg to about 60 mg of the free base compound,

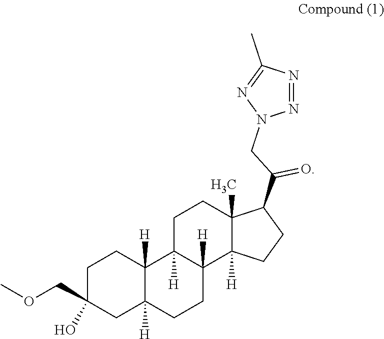

16. A method of treating irritable bowel syndrome (IBS) in a subject in need thereof, comprising administering a therapeutically effective amount of Compound (1), or a pharmaceutically acceptable salt thereof,

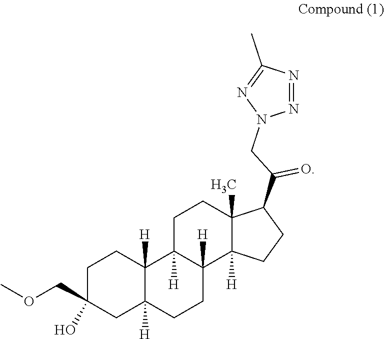

17. A method of treating irritable bowel syndrome (IBS) in a subject in need thereof, comprising administering from about 20 mg to about 60 mg of Compound (1), or a pharmaceutically acceptable salt of Compound (1) at a dose equivalent of from about 20 mg to about 60 mg of the free base compound,

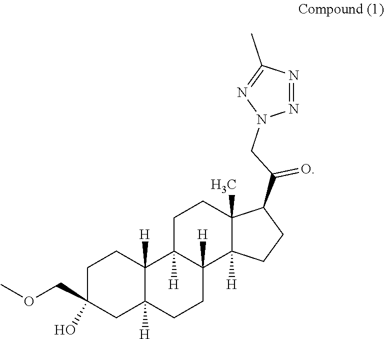

18. A method of treating a gastrointestinal (GI) disease or condition in a subject in need thereof, comprising administering a therapeutically effective amount of Compound (1), or a pharmaceutically acceptable salt thereof, 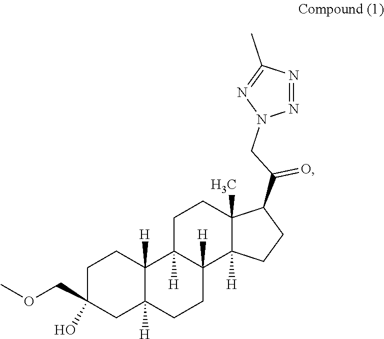
wherein the subject is being treated for a CNS-related disorder.

19. A method of treating irritable bowel syndrome (IBS) in a subject in need thereof, comprising administering a therapeutically effective amount of Compound (1), or a pharmaceutically acceptable salt thereof, 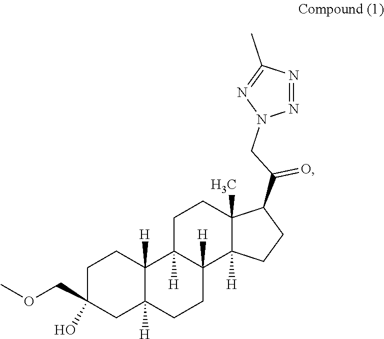
wherein the subject is being treated for a CNS-related disorder.
